(19) United States
(12) Patent Application Publication
(10) Pub. No.: US 2022/0284643 A1
(43) Pub. Date: Sep. 8, 2022

(54) METHODS OF ESTIMATION-BASED SEGMENTATION AND TRANSMISSION-LESS ATTENUATION AND SCATTER COMPENSATION IN NUCLEAR MEDICINE IMAGING

(71) Applicants: Abhinav Kumar Jha, St. Louis, MO (US); Ziping Liu, St. Louis, MO (US); Hae Sol Moon, St. Louis, MO (US); Zitong Yu, St. Louis, MO (US); Md Ashequr Rahman, St. Louis, MO (US)

(72) Inventors: Abhinav Kumar Jha, St. Louis, MO (US); Ziping Liu, St. Louis, MO (US); Hae Sol Moon, St. Louis, MO (US); Zitong Yu, St. Louis, MO (US); Md Ashequr Rahman, St. Louis, MO (US)

(73) Assignee: Washington University, St. Louis, MO (US)

(21) Appl. No.: 17/187,727

(22) Filed: Feb. 26, 2021

(51) Int. Cl. G06T 11/00; G06K 9/00; G06T 7/11; G06T 7/00; A61B 6/03; A61B 6/00

(52) U.S. Cl. CPC G06T 11/005; G06K 9/00201; G06K 9/00147; G06T 7/11; G06T 11/008; G06T 7/0012; A61B 6/037; A61B 6/5247; A61B 6/5282; G06K 2209/05; G06T 2207/10108; G06T 2207/10104; G06T 2207/20081; G06T 2211/424; G06T 2207/30096

(57) ABSTRACT

Among the various aspects of the present disclosure is the provision of methods for estimation-based segmentation of nuclear medicine images, as well as methods of transmission-less attenuation and scatter compensation of nuclear medicine images.

### CROSS-REFERENCE TO RELATED APPLICATIONS

This application claims the benefit of priority from U.S. Provisional Application No. 62/981,818, the content of which is incorporated by reference in its entirety.

### STATEMENT REGARDING FEDERALLY SPONSORED RESEARCH OR DEVELOPMENT

This invention was made with government support under EB024647 awarded by the National Institutes of Health. The government has certain rights in the invention.

### MATERIAL INCORPORATED-BY-REFERENCE

Not applicable.

### FIELD OF THE DISCLOSURE

The present disclosure generally relates to nuclear medicine imaging methods. In particular, the present disclosure relates to methods for estimation-based image segmentation, as well as methods of transmission-less attenuation and scatter compensation of nuclear medicine images.

### BACKGROUND OF THE DISCLOSURE

Attenuation and scatter are image-degrading effects in nuclear medicine imaging methods, such as PET and SPECT imaging. Compensating for these image-degrading processes would enhance the effectiveness of the quantification and visual interpretation of nuclear medicine images. However, attenuation and scatter compensation (ASC) typically makes use of a transmission scan, such as a CT scan, to produce an attenuation map used in ASC. The acquisition of CT scans leads to increased dose for the patient, increased scanning costs, longer acquisition times, and patient discomfort. The use of separate modalities introduces the risk of misalignment between the nuclear medicine and CT imaging data, resulting in the creation of false defects within the images.

In addition, segmentation of nuclear medicine images is used to implement a variety of tasks, such as PET-based radiotherapy planning and quantification of radiomic and volumetric features from nuclear medicine images. However, segmentation of nuclear medicine images is challenging for numerous reasons, such as partial-volume effects (PVEs). PVEs in PET imaging typically arise from two sources: limited spatial resolution of the imaging system and finite voxel size of the reconstructed image. The limited spatial resolution may lead to blurred tissue boundaries, and the finite voxel size may result in voxels containing a mixture of different tissues, also referred to herein as tissue-fraction effects (TFEs). To accurately account for TFEs, a continuous estimate of the tissue-fraction area within each pixel may be desired.

Other objects and features will be in part apparent and in part pointed out hereinafter.

### SUMMARY

In one aspect, a computer-implemented method for segmenting a nuclear medicine image is disclosed that includes transforming a nuclear medicine image dataset into a segmented nuclear medicine image dataset using a deep learning network, in which the segmented nuclear medicine image dataset includes a plurality of voxels. Each voxel is associated with at least one voxel volume fraction, and each voxel volume fraction is indicative of a fraction classified as one tissue type within each voxel. In some aspects, the deep learning network is configured to minimize a binary cross-entropy (BCE) of a Bayesian cost function to estimate the posterior mean of the one tissue type within each voxel. In some aspects, the deep learning network includes an autoencoder-decoder architecture. In some aspects, the method further includes training the deep learning network using a training dataset comprising a plurality of segmented MRI images and a corresponding plurality of segmented nuclear medicine images. In some aspects, the nuclear medicine image is selected from the group consisting of a PET image and a SPECT image.

In another aspect, a computer-implemented method for performing transmission-less attenuation and scatter compensation (ASC) on a nuclear medicine image is disclosed that includes reconstructing a scatter window dataset corresponding to the nuclear medicine image to obtain a preliminary attenuation map, transforming the preliminary attenuation map to a final estimated attenuation map by segmenting the preliminary attenuation map using a deep learning network, and reconstructing the nuclear medicine image based on a photopeak window associated with the nuclear medicine image in combination with the final estimated attenuation map. In some aspects, the deep learning network is configured to minimize a binary cross-entropy (BCE) of a Bayesian cost function to estimate the posterior mean of the one tissue type within each voxel of the preliminary attenuation map. In some aspects, the deep learning network includes an autoencoder-decoder architecture. In some aspects, the method further includes training, using the computing device, the deep learning network using a training dataset comprising a plurality of segmented MRI images and a corresponding plurality of segmented nuclear medicine images. In some aspects, the nuclear medicine image is selected from the group consisting of a PET image and a SPECT image.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a block diagram schematically illustrating a system in accordance with one aspect of the disclosure.

FIG. 2 is a block diagram schematically illustrating a computing device in accordance with one aspect of the disclosure.

FIG. 3 is a block diagram schematically illustrating a remote or user computing device in accordance with one aspect of the disclosure.

FIG. 4 is a block diagram schematically illustrating a server system in accordance with one aspect of the disclosure.

FIG. 5 is a schematic illustration of the workflow of the simulation performed in Example 1 below, in which structural boundaries of MR images are extracted with Freesurfer, and a DaT activity distribution map in relevant regions from clinical data (left image) is obtained. A highly realistic SPECT simulation is performed to obtain a simulated sonogram (center image) and a 3D OS-EM reconstruction (right image).

FIG. 6 contains a series of images comparing a SPECT image segmented using the disclosed 3D segmentation method to a ground-truth segmentation and segmented images obtained using several existing methods.

FIG. 7 contains a series of SPECT images comparing the disclosed 3D segmentation method to ground-truth segmentation for two subjects; upper slice shows an axially upper slice and lower slice shows axially lower slice of each subject's slice.

FIG. 8 contains a series of graphs comparing fDSC, fJSC, and HD obtained using the disclosed segmentation method as compared to four existing segmentation methods; separate segmentations were conducted for images with 2 mm voxel size (top row) and 4 mm voxel size (bottom row).

FIG. 9A contains a series of graphs comparing the accuracy of the SBR obtained using the different segmentation methods with SPECT images of 2 mm voxel size and (b) 4 mm voxel size.

FIG. 9B contains a series of graphs comparing the accuracy of the SBR obtained using the different segmentation methods with SPECT images of 4 mm voxel size.

FIG. 10 contains graphs comparing segmentation accuracy of the disclosed methods and several existing methods when the MR and SPECT images are misregistered; the left graph shows results with 2 mm voxel size and right graph shows results with 4 mm voxel size.

FIG. 11A is a flow chart showing the generation of a high-resolution tumor model.

FIG. 11B is a flow chart showing the generation of PET images with known tumor boundaries using the high-resolution tumor model of FIG. 11A.

FIG. 12 is a screenshot of a representative interface of the web-based application presented to readers for the 2-AFC test described in Example 2; (FOV: field of view).

FIG. 13 contains pairs of images of representative high-resolution tumor models (left) with different tumor features and simulated images (right) based on the tumor models; tumor locations in the simulated images are marked by red arrows.

FIG. 14 contains a series of images of simulated images (right) based on the tumor models that were incorrectly identified by at least 3 readers with confidence levels ≥3; tumor locations in the simulated images are marked by red arrows.

FIG. 15 contains a series of histograms of the distribution of confidence levels for correctly (upper row) and incorrectly (lower row) identified pair counts for four board-certified radiologists with specialization in nuclear medicine (left column), one nuclear-medicine physicist (center column), and all readers (right column).

FIG. 16A is a map of the ensemble-average bias using the disclosed method.

FIG. 16B is a graph comparing the pixel-wise EMSE between the true and estimated tumor-fraction areas using the methods annotated in FIG. 16A.

FIG. 16C is a graph comparing the area-EMSE between the measured and true tumor areas using the methods annotated in FIG. 16A.

FIG. 16D is a graph comparing Fuzzy Dice Similarity Coefficients between the true and estimated segmentations using the methods annotated in FIG. 16A.

FIG. 16E is a graph comparing the Fuzzy Jaccard Similarity Coefficients between the true and estimated segmentations using the methods annotated in FIG. 16A.

FIG. 16F is a graph comparing the Volume Similarity between the true and estimated segmentations using the methods annotated in FIG. 16A.

FIG. 16G is a graph comparing the Hausdorff Distance between the true and estimated segmentations using the methods annotated in FIG. 16A.

FIG. 17 is a series of images showing tumor contours delineated using the disclosed method (green) compared to the high-resolution ground-truth contour (red) and mU-net, another DL-based binary segmentation method (blue); zoomed contours are displayed for better visualization.

FIG. 18A is a graph summarizing the effects of tumor size on normalized pixel-wise EMSE from segmentations using the disclosed method, a U-net-Based method, and the MRF-GMM method.

FIG. 18B is a graph summarizing the effects of tumor size on normalized area EMSE from segmentations using the disclosed method, a U-net-Based method, and the MRF-GMM method.

FIG. 18C is a graph summarizing the effects of tumor size on fDSC from segmentations using the disclosed method, a U-net-Based method, and the MRF-GMM method.

FIG. 18D is a graph summarizing the effects of tumor size on fJSC from segmentations using the disclosed method, a U-net-Based method, and the MRF-GMM method.

FIG. 18E is a graph summarizing the effects of tumor size on VS from segmentations using the disclosed method, a U-net-Based method, and the MRF-GMM method.

FIG. 18F is a graph summarizing the effects of tumor size on HD from segmentations using the disclosed method, a U-net-Based method, and the MRF-GMM method.

FIG. 19A is a series of images summarizing a qualitative comparison between the PVEs-affected boundaries and the predicted tumor boundaries by the disclosed method.

FIG. 19B is a graph summarizing a quantitative comparison of % area overlap between the PVEs-affected boundaries and the predicted tumor boundaries by the disclosed method.

FIG. 20A is a schematic diagram illustrating a method for reconstructing an attenuation map in accordance with one aspect of the disclosure.

FIG. 20B is a graph summarizing a quantitative comparison of attenuation maps generated using the method illustrated in FIG. 20A, using Chang's method, and the true attenuation map; LP/RP and LC/RC de-note left/right putamen and caudate, respectively.

FIG. 20C contains a series of representative reconstructed SPECT images obtained using attenuation maps obtained as summarized in FIG. 20B.

FIG. 21 is a schematic diagram of SPECT system demonstrating the definition of a path.

FIG. 22A is a normalized RMSE plot of activity uptake in the caudate region as a function of iteration number for different configurations of energy window.

FIG. 22B is a normalized RMSE plot of activity uptake in the putamen region as a function of iteration number for different configurations of energy window.

FIG. 22C is a normalized RMSE plot of activity uptake in the caudate region as a function of iteration number for different binnings of energy window.

FIG. 22D is a normalized RMSE plot of activity uptake in the putamen region as a function of iteration number for different binnings of energy window.

FIG. 23A is an image of true activity.

FIG. 23B is an image of true activity and a reconstructed image using OS-LM-MLEM with LM data and the entire energy spectrum.

FIG. 23C is an image of true activity and a reconstructed image using OS-LM-MLEM with the entire energy spectrum but binned data with 2 energy bins.

FIG. 23D is an image of true activity and a reconstructed image using OS-LM-MLEM with LM data only from PP window.

FIG. 24 is a schematic diagram illustrating a disclosed method of differentiating the caudate, putamen, and GP structures in a SPECT image of the brain; the disclosed method delineates the regions accurately, providing for reliable quantification of regional uptakes.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

Typical segmentation algorithms used in nuclear medicine imaging are classification-based, i.e. they classify each pixel as belonging to one of several classes corresponding to different tissue types such as bone, tumor tissue, grey matter, and other tissue types. However, in medical imaging, a particular tissue may occupy a certain continuous-valued area/volume within a pixel/voxel of a given image. In some cases, the boundary of such tissue may occupy only a portion of the voxels defining the tissue boundary. Existing classification-based segmentation approaches do not provide for these partial volume effects.

In various aspects, an estimation-based segmentation method is provided that estimates the fractional area/volume that each tissue occupies within each pixel/voxel. The segmentation method disclosed herein enables segmenting low-resolution imaging data at high resolution. In addition, the disclosed segmentation method incorporates a physics-guided approach to account for blur due to system resolution while performing segmentation. Additional description of the estimation-based segmentation method is provided in Examples 1 and 2 below, as well as associated Appendices A and B, incorporated by reference herein.

In various other aspects, a process to reconstruct SPECT images such that they are able to compensate for attenuation and scatter even without the availability of a transmission scan, such as a CT scan, is disclosed. The disclosed method for transmission-less attenuation and scatter compensation yields equivalent performance to that yielded by an attenuation scattering compensation (ASC) technique that uses a transmission scan. Additional description of the transmission-less attenuation and scatter compensation method is provided in Example 3 below, as well as associated Appendix C, incorporated by reference herein.

Although the segmentation methods and attenuation/scatter compensation methods are disclosed in terms of specific nuclear medicine imaging modalities including, but not limited to, PET imaging and SPECT imaging, the methods disclosed herein are compatible with any nuclear medicine imaging method without limitation. Similarly, the disclosed segmentation and attenuation/scatter compensation methods may be used to enhance the quality of nuclear medicine images of any region of interest without limitation. Non-limiting examples of regions of interest suitable for nuclear medicine imaging data processing using the disclosed methods include brain, chest, heart, lungs, neck, abdomen, and any other suitable body region.

In various aspects, the disclosed segmentation methods and attenuation/scatter compensation methods are implemented using various computing systems and devices as described below.

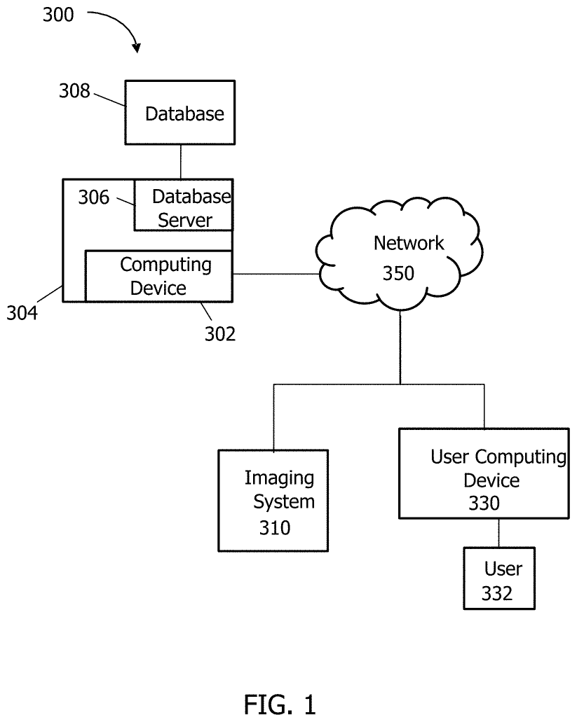

FIG. 1 depicts a simplified block diagram of a computing device for implementing the methods described herein. As illustrated in FIG. 1, the computing device 300 may be configured to implement at least a portion of the tasks associated with the disclosed method using the imaging system 310 including, but not limited to: operating the imaging system 310 to obtain nuclear medicine imaging data. The computer system 300 may include a computing device 302. In one aspect, the computing device 302 is part of a server system 304, which also includes a database server 306. The computing device 302 is in communication with a database 308 through the database server 306. The computing device 302 is communicably coupled to the imaging system 310 and a user-computing device 330 through a network 350. Network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user-computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

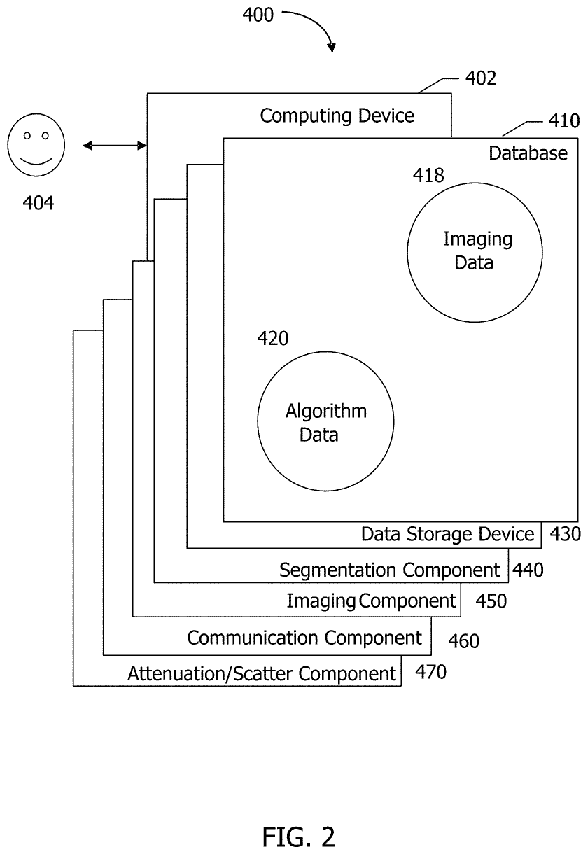

In other aspects, the computing device 302 is configured to perform a plurality of tasks associated with obtaining nuclear medicine images. FIG. 2 depicts a component configuration 400 of computing device 402, which includes database 410 along with other related computing components. In some aspects, computing device 402 is similar to computing device 302 (shown in FIG. 1). A user 404 may access components of computing device 402. In some aspects, database 410 is similar to database 308 (shown in FIG. 1).

In one aspect, database 410 includes imaging data 418 and algorithm data 420. Non-limiting examples of suitable algorithm data 420 include any values of parameters defining the analysis of nuclear medicine imaging data, such as any of the parameters from the equations and/or machine learning model described herein.

Computing device 402 also includes a number of components that perform specific tasks. In the exemplary aspect, computing device 402 includes data storage device 430, segmentation component 440, imaging component 450, communication component 460, and segmentation/scatter component 470. Data storage device 430 is configured to store data received or generated by computing device 402, such as any of the data stored in database 410 or any outputs of processes implemented by any component of computing device 402. Imaging component 450 is configured to operate or produce signals configured to operate, an imaging device to obtain nuclear medicine imaging data, and to reconstruct the nuclear medicine image (PET or SPECT image) based on the nuclear medicine imaging data.

Communication component 460 is configured to enable communications between computing device 402 and other devices (e.g. user computing device 330 and imaging system 310, shown in FIG. 1) over a network, such as network 350 (shown in FIG. 1), or a plurality of network connections using predefined network protocols such as TCP/IP (Transmission Control Protocol/Internet Protocol).

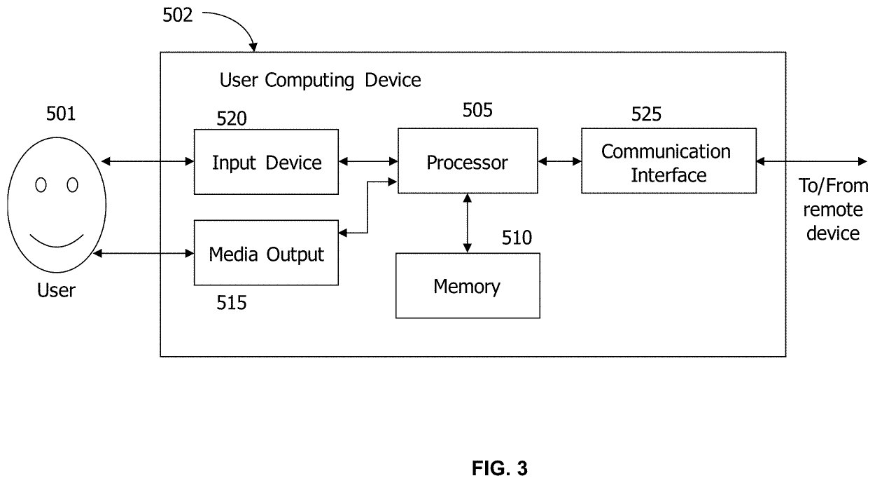

FIG. 3 depicts a configuration of a remote or user-computing device 502, such as user computing device 330 (shown in FIG. 1). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light-emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

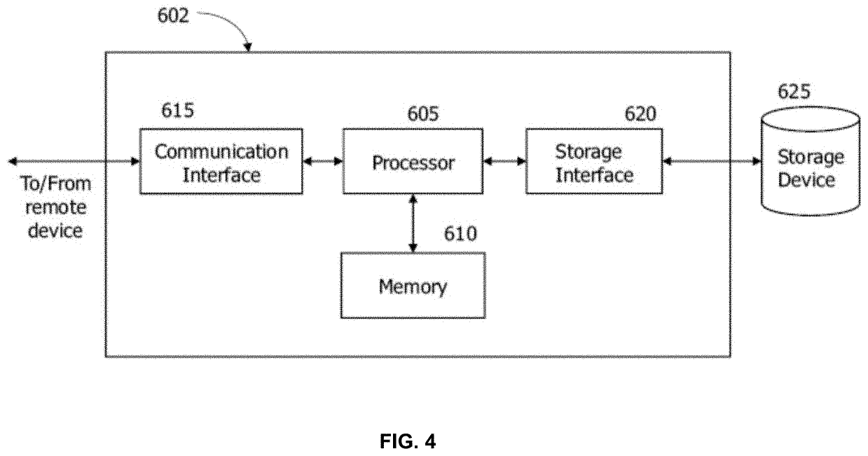

FIG. 4 illustrates an example configuration of a server system 602. Server system 602 may include, but is not limited to, database server 306 and computing device 302 (both shown in FIG. 1). In some aspects, server system 602 is similar to server system 304 (shown in FIG. 1). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 1) or another server system 602. For example, communication interface 615 may receive requests from user computing device 330 via a network 350 (shown in FIG. 1).

Processor 605 may also be operatively coupled to a storage device 625. Storage device 625 may be any computer-operated hardware suitable for storing and/or retrieving data. In some aspects, storage device 625 may be integrated in server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 3) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are example only, and are thus not limiting as to the types of memory usable for storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may further include: sensor data, image data, video data, telematics data, authentication data, authorization data, security data, mobile device data, geolocation information, transaction data, personal identification data, financial data, usage data, weather pattern data, “big data” sets, and/or user preference data. In some aspects, data inputs may include certain ML outputs.

In some aspects, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically ML methods and algorithms directed toward reinforcement learning may receive a user-defined reward signal definition, receive a data input, utilize a decision-making model to generate an ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

As will be appreciated based upon the foregoing specification, the above-described aspects of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving media, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

In one aspect, a computer program is provided, and the program is embodied on a computer-readable medium. In one aspect, the system is executed on a single computer system, without requiring a connection to a server computer. In a further aspect, the system is being run in a Windows® environment (Windows is a registered trademark of Microsoft Corporation, Redmond, Wash.). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independently and separate from other components and processes described herein. Each component and process can also be used in combination with other assembly packages and processes. The present aspects may enhance the functionality and functioning of computers and/or computer systems.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following examples illustrate various aspects of the disclosure.

### Example 1: 3D Automated Segmentation Method for DaT-Scan SPECT Images Using Priors Derived from MR Images

The following example describes a method of automated image segmentation that made use of MR images obtained from previously acquired patient populations to provide accurate delineation of regions within SPECT brain images at high resolution. The method described below incorporated information from prior MR images and was further guided by the physics of the SPECT imaging system to inherently account for at least two sources of partial volume effects in SPECT images: 1) limited system resolution, and 2) tissue-fraction effects. The method was developed in the context of quantitative dopamine-transporter (DaT)-scan SPECT imaging.

The automated image segmentation method was evaluated both qualitatively and quantitatively using highly realistic simulation studies. The method yielded accurate boundaries of the caudate, putamen and globus pallidus regions, provided reliable estimates of the specific binding ratios of these regions, and significantly outperformed several commonly used existing segmentation methods.

To address the need for improved methods for segmentation of DaTScan brain SPECT images, we recognize that a major barrier to segmenting DaT-scan SPECT images into caudate, putamen, and GP is the lack of clear boundaries between these regions in the SPECT images. This arises due to the partial volume effects (PVEs) in SPECT images. These PVEs have two sources: limited system resolution and tissue fraction effects. Clinical brain SPECT systems typically have resolution of the order of about 4 mm. Thus, structures such as caudate, putamen and globus pallidus, which have much lower separation between each other are difficult to separate on SPECT images. In this context, these regions are separately visible on MR images, where they can be easily delineated. MR images of existing patient populations can thus provide a prior distribution of the boundaries of these regions.

The other source of PVEs in SPECT images is tissue-fraction effects (TFEs). This arises because of the reconstruction of the image over a finite-sized voxel grid, due to which it is likely that a voxel contains more than one region. While this is generally an issue in medical imaging, the effect is more prominent in SPECT images due to the larger voxel sizes. Conventional segmentation methods are designed as classifiers, in that they classify a voxel as belonging to a certain region. Thus, they are inherently unable to account for the TFEs.

To address this issue in the context of segmenting oncological 2-D PET images an estimation-based segmentation method has been previously proposed. The method estimates the posterior mean of the fraction tumor area within each pixel using a learning-based approach. The technique requires a prior distribution of the fractional tumor areas, which are obtained from realistic simulations that are guided by clinical data. We now propose to extend this technique in the context of segmenting multiple regions, namely the left and right caudate, putamen and globus pallidus, from 3-D SPECT images. The basic idea is that for each voxel, we estimate the fractional volume that a particular region occupies in that pixel. The prior distribution for these fractional volumes is obtained from existing populations of MR images. The disclosed method uses this prior distribution to estimate the posterior mean of the fractional volumes of the caudate, putamen, and globus pallidus regions from the DaT-scan SPECT images. These estimates yield the segmentations of these regions over a continuous non-voxelized grid, analogous to segmentation of a high-resolution image.

### Methods

### Problem Formulation

Let ƒ(r) describe the dopamine tracer distribution within the brain for the scanned patient, where r is a 3D vector denoting spatial location. Consider a SPECT system that images this 3D tracer distribution and yields sinogram data. The sinogram data is input to a reconstruction method, yielding a reconstructed image \hat{ƒ} consisting of M voxels. Denote the process of obtaining the reconstructed image from the original tracer distribution by the operator θ: [symbol] ₂( [symbol] ³)→ [symbol] ^{M}. Our objective is to segment the reconstructed image into K regions, in this case, the left and right caudate, putamen, and GP regions, with the rest of the region labeled as the background. Typically this is performed by labeling each voxel in the reconstructed image to one region. However, this segmentation is unable to account for tissue-fraction effects. Thus, we instead frame the segmentation problem as estimating the fractional volume occupied by the k^{th} region in each voxel of the reconstructed image \hat{ƒ}.

Let the support of the k^{th} region be given by ϕₖ(r). Mathematically,

$${ϕ}_{k} ( r ) = \{ \begin{matrix}1 , & if at location r , region k is present . \\ 0 , & otherwise\end{matrix} . \qquad Eqn . ( 1 )$$

Also, let the distribution of tracer uptake within the support of each region be given by ƒₖ(r). Then ƒ(r) can be described as follows:

$$f ( r ) = {\sum }_{k = 1}^{K} {f}_{k} ( r ) {ϕ}_{k} ( r ) \qquad Eqn . ( 2 )$$

Next, define the voxel function ψₙ(r) as:

$${ψ}_{m} ( r ) = \{ \begin{matrix}1 , & if r lies within the {m}^{th} voxel \\ 0 , & otherwise\end{matrix} \qquad Eqn . ( 3 )$$

Let V denote the total volume of each voxel. The fractional volume occupied by the k^{th} region in the m^{th} voxel, denoted by v_{ideal,k,m}, is given by:

$$\text{?} = \frac{1}{V} \int {ϕ}_{k} ( r ) {ψ}_{m} ( r ) {d}^{3} r \qquad Eqn . ( 4 )$$

$$\text{?} \text{indicates text missing or illegible when filed}$$

Estimating the fractional volumes within each voxel thus requires estimating the values of ϕₖ(r) from \hat{f}. This is an ill-posed problem due to the null functions of the Θ operator. One way to alleviate the ill-posedness issue is to incorporate prior distribution about ϕₖ(r). However, this requires access to the distribution of ƒ(r), or at least ϕₖ(r), at infinitely high resolution, which is practically not possible to obtain.

A more practical solution is to learn the distribution of the support ϕₖ(r) at a relatively high resolution from previously acquired MR images of different patients. More specifically, the anatomical structures of the caudate, putamen and GP regions are more easily distinguishable on MR images compared to SPECT images, owing to the higher resolution of MR images. The MR images can thus be segmented to yield the support of these regions at a higher resolution than in SPECT images. Thus, from previously acquired MR images, a distribution of the support of these regions can be obtained.

Denote the MR image by an N-dimensional vector f_{MR}, where N>M. The voxel function for this image is denoted as ψₘ^{MR}(r). Assume that this image has been segmented into K distinct regions. For each region, we define an N-dimensional vector ϕₖ^{MR}(r), which denotes the mask for that region and is defined as follows:

$${ϕ}_{k , i}^{MR} = \{ \begin{matrix}1 , & if voxel i is assigned to region k \\ 0 , & otherwise\end{matrix} \qquad Eqn . ( 5 )$$

A high-resolution estimate of the fractional volume occupied by the k^{th} region in the m^{th} voxel of the SPECT image, denoted by v_{k,m}, is given by

$${v}_{k , m} = \frac{{\sum }_{m = 1}^{M} {ϕ}_{k}^{MR} {ψ}_{m}^{MR}}{V} \qquad Eqn . ( 6 )$$

We frame our problem as the task of estimating {v_{k,m}, k=1, 2, . . . K, m=1, 2, . . . M} from the reconstructed SPECT images.

### Estimation Technique

Denote the estimate of v_{k,m} by \hat{v}_{k,m}. Denote the vector {v_{k,m}, m=1, 2, . . . M} by vₖ, and the estimate of vₖ by \hat{v}ₖ. To computer \hat{v}ₖ from the reconstructed SPECT image \hat{f}, we first need to define a cost function. The values of v_{k,m} and \hat{v}_{k,m} lie between 0 and 1. Thus, if we treat these values as probabilities, the binary cross-entropy (BCE) between v_{k,m} and \hat{v}_{k,m} automatically incorporates this constraint on the range of these values. Therefore, the BCE loss, denoted by l_{BCE}(v_{k,m}, \hat{v}_{k,m}), and given by the equation below is chosen as the basis of the cost function.

l_{BCE}(v_{k,m},\hat{v}_{k,m})=v_{k,m} log(\hat{v}_{k,m})+(1−v_{k,m})log(1−\hat{v}_{k,m}) Eqn. (7)

The cost function, denoted by C(vₖ, \hat{v}ₖ), minimizes the negative of the BCE between v_{k,m} and \hat{v}_{k,m} over all M voxels, averaged over the ensemble of true values v_{k,m} and the noise in the imaging process. Here we note that the number of voxels in the VOIs is much lower than the number of voxels in the background. To account for this imbalance, we assign different weights to the voxels in the VOIs and the background. Denote the weight assigned to the k^{th} region by wₖ. The cost function is then given by:

$$C ( {v}_{k} , \text{?} ) = - {\sum }_{m = 1}^{M} \int \int p ( \hat{F} , {v}_{k , m} ) {w}_{k} \text{?} ( {v}_{k , m} , \text{?} ) {dv}_{k , m} {d}^{M} \hat{f} \qquad Eqn . ( 8 )$$

$$\text{?} \text{indicates text missing or illegible when filed}$$

We use Bayes theorem to expand p(\hat{f},v_{k,m}) and replace the BCE expression from Eqn. (7) in the cost function of Eqn. (8). This yields:

$$\begin{matrix}C ( {v}_{k} , \text{?} ) = - {\sum }_{m = 1}^{M} \int p ( \text{?} ) \int \text{?} \\ = - {\sum }_{m = 1}^{M} \int p ( \text{?} ) \int \text{?} \\ = {\sum }_{m = 1}^{M} \int p ( \text{?} ) [ ( log ( \text{?} ) - log ( \text{?} ) ) \int \text{?}\end{matrix} \qquad Eqn . ( 9 )$$

$$\text{?} \text{indicates text missing or illegible when filed}$$

The term p(\hat{f}) is always positive. Thus, the cost function to be minimized by setting

$$\frac{\partial }{\partial \text{?}} [ \text{} \{ log ( \text{?} ) - log ( 1 - \text{?} ) \} \int p ( {v}_{k , m} \vert \hat{f} ) {v}_{k , m} {dv}_{k , m} + log ( 1 - \text{?} ) ] = 0 \qquad Eqn . ( 10 )$$

$$\text{?} \text{indicates text missing or illegible when filed}$$

The solution is given by

\hat{v}_{k,m}*=∫p(v_{k,m}|\hat{f})v_{k,m}dv_{k,m} Eqn. (11)

Eqn. (11) above is an expression for the posterior mean estimate of v_{k,m}.

Thus, denoting the point at which the cost function is minimized by \hat{v}_{k,m}*, we can obtain:

\hat{v}_{k,m}*=∫p(v_{k,m}|\hat{f})v_{k,m}dv_{k,m} Eqn. (12)

The method disclosed above estimates the probability of each voxel belonging to a certain class by integrating the volumetric fraction of each voxel in the MR prior into SPECT space. This allows the model to learn from a continuous prior and perform segmentation into low resolution and voxelated SPECT images. Thereby accounting for TFE in the process.

### Implementation

An autoencoder-decoder-based architecture was implemented to minimize the cost function defined in Eqn. (7). The auto-encoder was input the 3D SPECT images and corresponding MR-defined ground-truth masks. We followed this approach instead of conducting slice-by-slice segmentation or patch-based training to provide the maximal amount of global contextual information per patient image. We used a weighted voxel-wise softmax with cross-entropy loss function for multiclass segmentation. We empirically determined that a weight of 1 for the background and a weight of 2 for the six volumes of interest were optimal for conducting segmentation. This weight ratio of 2:1 suggested that even though the 6 VOIs contained more information to perform the segmentation task compared to the background, the background also contained important information. This reinforced the notion that the shape and volume of ROIs were also related to the shape and volume of the background, further motivating the incorporation of contextual information. The method was implemented on state-of-art NVIDIA Titan RTX GPU with 24 GB of memory and NVIDIA V100 with 32 GB of memory, allowing effective training with 3D images.

### Evaluation of the Disclosed Method

Evaluation of the disclosed method requires access to DaT-scan SPECT images where the MR-defined boundaries of the VOIs are known. Ideally, this would require a setup where both the MR and DaT-scan SPECT images for a certain set of patients were acquired. Another way to perform this evaluation is through the use of realistic simulation studies, where clinical MR images are used to generate realistic SPECT images by modeling the physics of SPECT imaging. This process inherently ensures that the MR-defined ground-truth boundaries of the VOIs in the generated SPECT images are known. In this section, we describe the process we followed to rigorously evaluate the disclosed method using this simulation-based approach.

### Generating Realistic SPECT Images

A total of 600 T1-weighted MR images from the Open Access Series of Imaging Studies (OASIS) 3 and Alzheimer's Disease Neuroimaging Initiative (ADNI) databases were used. These images were segmented into grey matter, white matter, caudate, putamen and globus pallidus for both hemispheres using the Freesurfer software. The delineation was performed in Montreal Neurological Institute (MNI) space into 256×256×256 voxels with voxel size of 1 mm. The resulted delineations of MR images yielded 600 anatomical templates of the brain. For each template, the in vivo distribution of DaT activity ratios in the different regions of the brain was sampled to determine the DaT activity ratios in these regions. This process ensured the variability of activity for different subjects. At the end of this process, we had a digital population of 600 patients with anatomical templates directly obtained from clinical data and tracer distributions guided by clinical data.

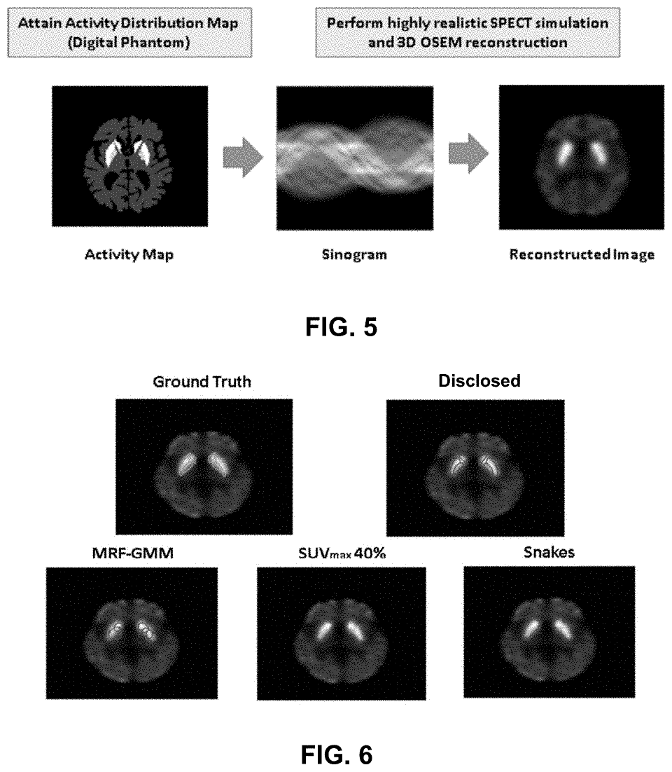

This ground-truth tracer distribution was used to create realistic DaT-scan SPECT images. A SPECT system modeling a DaT-scan study with 123-Ioflupane tracer and a low-energy high-resolution (LEHR) collimator was simulated. System parameters were similar to a GE Discovery 670 (GE Healthcare, Haifa, Israel). The simulation process modeled the various aspects of SPECT imaging, including the finite extent of the collimator, the finite energy and spatial resolution of the detectors and the process of photon attenuation. Using this simulation process, 100 million photon counts were generated for each of the 600 ground-truth tracer distributions defined above, simulating an almost noiseless data acquisition for each digital phantom. The number of total counts was scaled down to clinically realistic value of 2 million counts to which Poisson noise was added. The noisy projection data were reconstructed using an iterative 3D order-subsets expectation-maximization (OS-EM) algorithm with 4 iterations and 8 subsets. The OSEM technique incorporated attenuation compensation and the collimator-detector response. The reconstructed images were of dimensions 128×128×128 with two voxel sizes, namely 2 mm and 4 mm, to study the performance of the segmentation method for these two clinically relevant voxel sizes. The overall process to generate the SPECT images is summarized in FIG. 5.

### Training and Evaluation Strategy

The 600 generated 3D images were divided into two sets of 500 training images and 100 testing images for both the voxel sizes.

The method disclosed above was compared with several commonly used methods that have been used for segmenting SPECT images. These included a Markov random fields-Gaussian mixture model (MRF-GMM)-based method, an active contours-based technique, a thresholding-based technique, namely 40% SUV-max thresholding and finally a state-of-the-art U-net-based technique. For the first three segmentation methods, a manual input, which could be a seed pixel or an initial boundary estimate, was provided to the segmentation technique. Also, the 40% SUV-max and Snakes were only able to segment the striatal and pallidal region as a whole and were not able to separately segment caudate, putamen and globus pallidus. Thus, the resulting entire segmentation from these methods remained the same in segmenting for caudate, putamen and globus pallidus and was compared separately with the ground-truth mask of individual regions. Each of the compared methods, including the U-net-based method, assigned each pixel as belonging to a specific region, unlike the disclosed method. The disclosed method and the U-net-based method did not require any user inputs, and thus were fully automated.

The disclosed method was qualitatively and quantitatively evaluated and compared with these commonly used segmentation methods using metrics that evaluated spatial-overlap and shape similarity. Also, since the objective of segmenting the regions is to estimate the specific binding ratios (SBRs) for the different segmented regions, we evaluate the disclosed method on how well it assisted in this task.

For qualitative evaluation, the segmentation boundaries using the different methods were visually compared to the ground-truth boundary. For quantitative evaluation, note that the output from the segmentation method is an estimate of the tumor fraction area, which is a number between 0 and 1. Conventional segmentation methods typically assign a pixel to only a single class. For these methods, the spatial overlap is quantified using dice similarity coefficient and Jaccard similarity coefficient. Since our method yields a number between 0 and 1, we used the metric of fuzzy dice similarity coefficient (fDSC) and fuzzy Jaccard similarity coefficient (fJSC) to quantify visual overlap. Let TP, FP and FN denote true positive, false positive and false negative respectively. Then the fDSC and fJSC values are given by the following equations:

$$fDSC = \frac{2 T P}{2 T P + F P + F N} \qquad Eqn . ( 13 )$$

$$FJSC = \frac{T P}{T P + F P + F N} \qquad Eqn . ( 14 )$$

The higher values indicate a more accurate segmentation. Also, the similarity in shapes between the true and estimated segmentations was quantified using the Hausdorff distance (HD). HD measures the maximum deviation where the predicted and true segmentation boundaries. Thus, lower values of HD represent higher shape similarity.

To evaluate the disclosed method on the task of SBR quantification in the caudate, putamen and globus pallidus, we calculated the mean uptake value within these regions using boundary as defined with the disclosed segmentation method. The mean uptake value within a manually-defined reference region, which in this case was a box in the occipital lobe. The SBR for the different regions, denoted as SBR, was estimated as follows:

$$= \frac{E s t i mated mean activity in segmented regions}{E s t i mated mean activity in reference region} \qquad Eqn . ( 15 )$$

The true SBR was obtained from the ground-truth tracer distribution and using the true definition of the boundary. The normalized bias between the true and estimated SBR values was computed to quantify the accuracy in computing SBR.

### Results

### Qualitative Evaluation

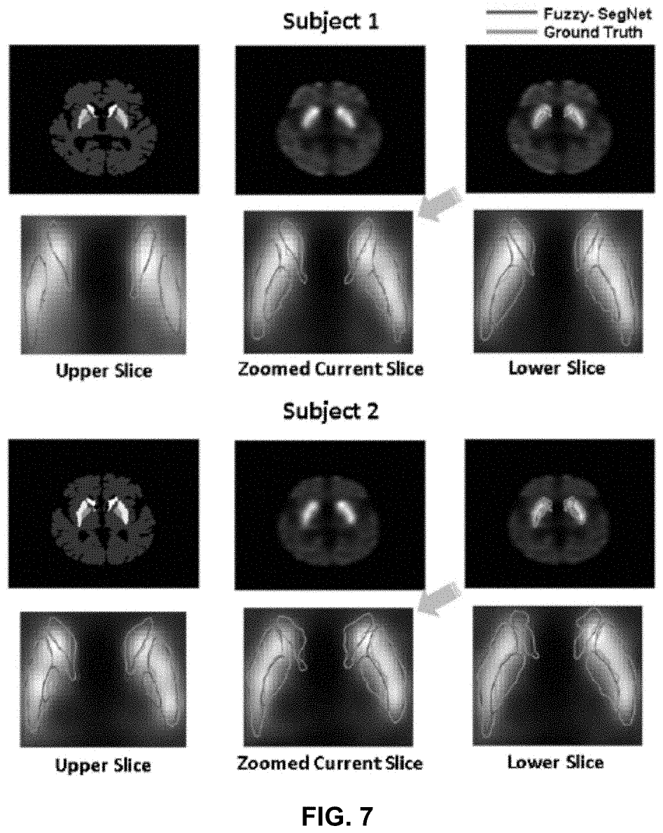

The segmentation boundaries estimated using the different methods for 2 mm voxel size SPECT images are shown in FIG. 6. The disclosed method closely matched the ground-truth boundary. This is shown more clearly using the zoomed-in results shown in FIG. 7. These results further illustrate the challenging nature of the segmentation task, as due to PVEs, the boundary between the different regions is highly blurred. As a result of this, the 40% SUV-max and active-contours methods segmented the striatal and pallidal regions but were unable to segment the various regions separately. The MRF-GMM technique could segment the individual regions but the overlap between the predicted and the ground truth was only partially congruent. In contrast, the disclosed method yielded accurate segmentation of all the individual regions.

### Quantitative Evaluation

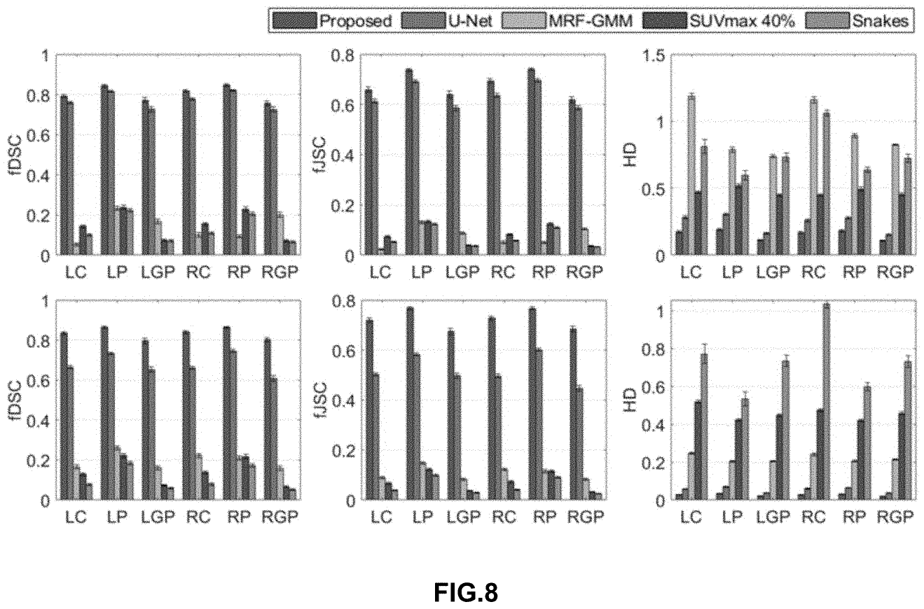

The top row in FIG. 8 shows that for the voxel size of 2 mm, the disclosed method outperformed other segmentation methods on the basis of the DSC, JSC, HD metrics, respectively. Also, the disclosed method yielded a DSC close to 0.8, thus indicating an accurate segmentation. While the existing methods yielded significantly higher chance of false segmentation for the caudate and the globus pallidus regions, the disclosed method did not suffer from this issue. Also, in the qualitative results, we had observed that MRF-GMM was able to segment individual regions. However, we see that this method yielded the lowest segmentation accuracy in terms of DSC, JSC and HD metrics.

Similar results were seen for the reconstructed images with voxel size of 4 mm, as shown at the bottom row in FIG. 8. In fact, the values of the metrics of DSC, JSC and HD remained relatively unchanged in going from 2 mm voxel size to 4 mm voxel size. This indicates the relative insensitivity of the method to change in voxel sizes, which we assess is due to the ability of the method to account for tissue-fraction effects.

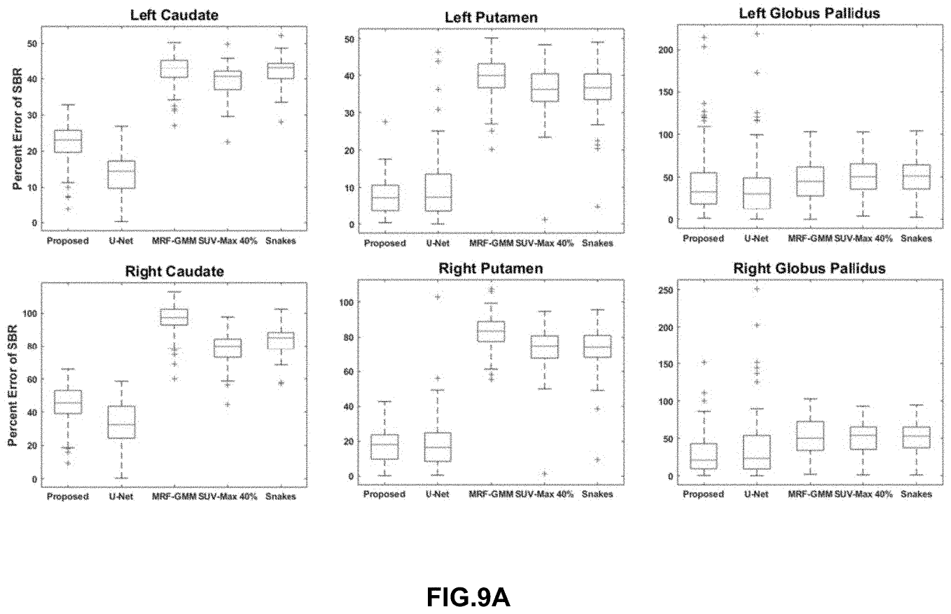

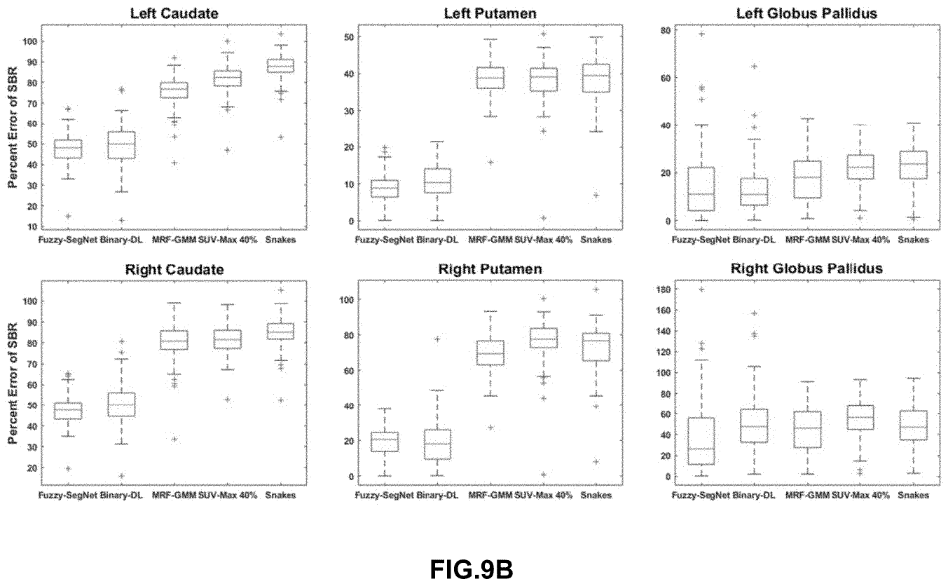

As illustrated in FIGS. 9A and 9B, the disclosed method sometimes yielded the most accurate or the second most accurate SBR values for both 2 mm and 4 mm voxel sizes.

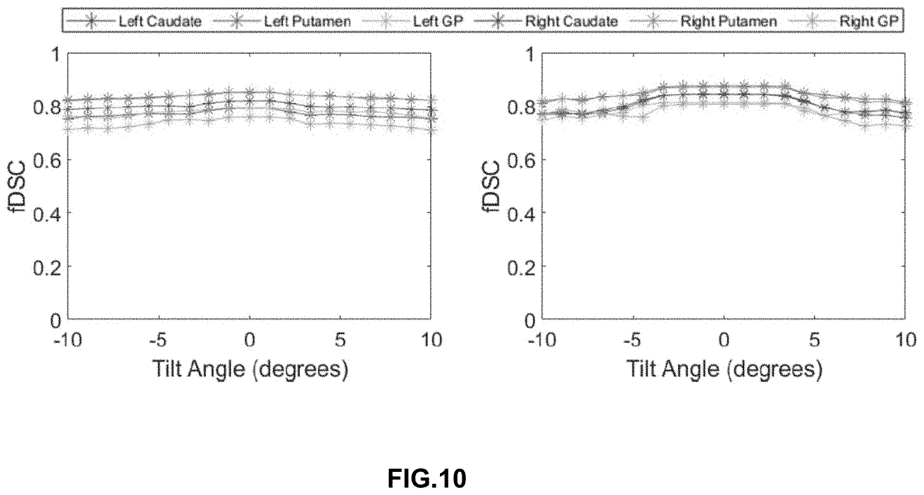

The accuracy of the disclosed method when misalignment between MR and SPECT images is introduced is shown in FIG. 10. The disclosed method maintained high accuracy without loss in performance between −4 and 4 degrees of misregistration. Also, with larger degrees of misregistration, the disclosed method yielded reliable accuracy for both 2 mm and 4 mm voxel sizes without significant decrease in performance.

### Discussion

The results of the above experiments demonstrate the efficacy of the disclosed method in reliably segmenting the caudate, putamen, and globus pallidus regions from DaT-scan SPECT images. The ability of segment the small-sized globus pallidus region, in particular, is distinctive and particularly useful, as the globus pallidus is visually almost impossible to demarcate on the SPECT images. This result opens up an important research frontier on analyzing the physiological characteristics of globus pallidus region in patients with Parkinson's disease (PD). It is well known that the globus pallidus plays an important role in the movement-related direct and indirect pathways of basal ganglia. There has been interest in studying the plasticity of the nigro-pallidal pathway and quantification of the DaT uptake in the pallial regions may provide new insights on this plasticity. Several post-mortem studies have shown evidence of DaT uptake in the pallidal regions. DaT-scan SPECT studies provide a mechanism to study these effects in vivo but these studies are hindered by the lack of tools to segment the pallidal regions on the SPECT images. The results of these experiments demonstrate that the disclosed method can help to address this challenge. Additionally, the disclosed method also provides an accurate delineation of the caudate and putamen regions in the SPECT images. This result provides tools to conduct shape analysis of these regions, which may also lead to new biomarkers for diagnosis and measuring severity of Parkinson's disease.

Previous methods that register the SPECT images with MR images have been developed to separately segment the caudate and putamen regions from DaT-scan SPECT images but not the globus pallidus. However, these previous approaches require that for a given SPECT image of a patient, the corresponding MR image be available, which may typically not be the case. Even if an MR image is available for the same patient, it may be from a different time point. While there has been some recent progress in the area of developing simultaneous SPECT/MR systems, no clinical simultaneous SPECT/MRI systems are currently available. The disclosed method does not require the corresponding MR image from the patient to perform the segmentation and works on stand-alone SPECT images.

The experiments described above focused on the task of segmenting the caudate, putamen and globus pallidus regions in the DaT-scan images and considered the rest of the region as background. However, post-mortem studies indicate DaT uptake in other regions in the basal ganglia such as the nucleus accumbens and substantia nigra. In addition, the globus pallidus can itself be separated into two parts, namely the internal and the external globus pallidus. In some aspects, the disclosed segmentation method may be extended to provide for segmenting other regions in the basal ganglia and/or segmenting the globus pallidus into two sub-regions from DaT-scan images.

Although U-net-based segmentation methods also learn anatomical variability and blurring due to PVE, it does not account for the TFE for which the disclosed method compensates. As the voxel sizes in SPECT images increase, so does the TFE. Thus, the disclosed method performs better than other segmentation methods especially when the voxel size is larger. This feature of the disclosed segmentation explains the increase in DSC and JSC differences between the disclosed method and other segmentation methods for images with 4 mm voxel size. With the increase of voxel size from 2 mm to 4 mm, the DSC of disclosed method remained consistent while that of the m-Unet segmentation method decreased >10%. Because many clinical DaT SPECT scans are recommended and conducted with 4 mm voxel size, this feature of the disclosed method is particularly useful. Also, the sensitivity of the disclosed method to misalignment between the SPECT and MR images demonstrated that the disclosed method maintained reliable accuracy even with the presence of misalignment.

PVEs have significant effect in activity quantification. Ideally, the activity difference between VOIs and the background should match perfectly with the ground truth boundaries shown in green in FIG. 7. The spill-over effects lead to significant loss of activity in VOIs. Thus, a more accurate segmentation does not necessarily lead to a more accurate activity uptake prediction due to such defection. This explains the observation that U-Net segmentation yielded lower % bias for left and right caudate than the disclosed method with 2 mm voxel size in FIG. 9A.

The results of these experiments demonstrate the need for partial volume compensation (PVC) directly to the reconstructed image prior to segmentation. The percent bias of activity ratio of the disclosed method is consistent with previously published results obtained without PVC, providing a basis for the expectation that after PVC, the percent bias of activity will decrease to less than 4 percent as shown in these previously-published results.

The results of these experiments demonstrate tremendous potential for shape and texture analysis studies of caudate, putamen and globus pallidus. The previous lack of separate segmentation of VOIs using previous methods limited such shape analysis to only striatum as a whole. The results of these experiments promise access to greater information in shape analysis of individual VOIs that could lead to the discovery of hidden features in DaTscan SPECT images that may correlate with the severity of PD and allow early detection of the neurodegenerative disease.

### Conclusion

Anatomical variability and PVEs have seriously limited accurate segmentation of separate ROIs in DaT SPECT images. Although the existing methods yielded decent estimation of the striatum as a whole, the PVEs have led to the inability and poor performance of discrete segmentation which have constrained the study of the shape analysis, texture analysis and activity quantification of caudate, putamen and GP individually. The disclosed estimation method described above makes use of priors derived from MR images and has proven to be highly effective in DaT SPECT images distorted by severe PVEs. The disclosed method incorporates the volumetric fraction of each voxel from MR priors and calculates the probability of each voxel belonging to a ROI, thus compensating sources of PVE, blurring and TFE. The segmentation results from the disclosed method yielded significantly improved qualitative and quantitative accuracy in DaT SPECT images.

### Example 2: Estimation-Based PET Segmentation Method that Accounts for Partial-Volume Effects

The following example describes a method of estimation-based PET image segmentation that yielded a posterior mean estimate of tumor-fraction area within each pixel and used these estimates to define a segmented tumor boundary. The method was implemented using an autoencoder and was evaluated in the context of segmenting tumors in oncological PET images of patients with non-small cell lung cancer using highly realistic simulation studies. The method was quantitatively evaluated in the context of segmenting primary tumors in 18F-fluorodeoxyglucose (FDG)-PET images of patients with non-small cell lung cancer.

High-resolution clinically realistic tumor models were generated using patient-data-derived tumor properties and intra-tumor heterogeneity was simulated using a stochastic lumpy model. The estimation-based segmentation method yielded superior tumor segmentation performance in these images, significantly outperformed commonly used existing segmentation methods, yielded reliable estimates of tumor-fraction areas, and was observed to be relatively insensitive to the partial-volume effects. Qualitatively, the method yielded accurate estimates of the continuous ground-truth tumor boundaries. Overall, the method yielded reliable tumor segmentation in PET images as validated using realistic simulation studies.

Tumor segmentation in oncological PET images is challenging, primarily due to the partial-volume effects that arise as a result of the low system resolution and the tissue-fraction effects. Conventional methods yield limited performance on the segmentation task because of the low resolution and the high noise. In addition, conventional methods are classification-based, i.e. they classify each pixel as either tumor or background, thus not accounting for the tissue-fraction effects. To address these issues, a method was developed that yields the posterior mean estimate of tumor-fraction area within each pixel and uses these estimates to define the segmented tumor boundary. This method was implemented using an autoencoder and evaluated in the context of segmenting tumors in oncological PET images of patients with non-small cell lung cancer using highly realistic simulation studies. In the following experiments, high-resolution clinically realistic tumor models were generated using patient-data-derived tumor properties and intra-tumor heterogeneity simulated using a stochastic lumpy model.

Realism of the PET images generated based on these tumors was investigated using a two-alternative-forced-choice study conducted by six trained readers. Accuracy around 50% for all readers demonstrated that the simulated images were highly realistic. The segmentation method disclosed in this example yielded superior tumor segmentation performance in these images, significantly outperformed commonly-used existing methods, yielded reliable estimates of tumor-fraction areas, and was relatively insensitive to the partial-volume effects. Qualitatively, the method yielded accurate estimates of the continuous ground-truth tumor boundaries. Overall, the method yielded reliable tumor segmentation in PET images as validated using realistic simulation studies.

Reliable oncological PET segmentation is required for several tasks such as PET-based radiotherapy planning and quantification of radiomic and volumetric features from PET images. However, PET segmentation is challenging due to at least several reasons, including, but not limited to, partial-volume effects (PVEs). The PVEs in PET imaging arise from two sources: limited spatial resolution of PET system and finite voxel size of the reconstructed image. The limited spatial resolution leads to blurred tumor boundaries, while the finite voxel size results in voxels containing a mixture of tumor and normal tissue.

Manual delineation is the most common PET segmentation technique, but this technique is both labor and time-intensive, suffers from intra- and inter-operator variability, and has limited accuracy because of the PVEs. Computer-aided segmentation methods have been developed to address these issues, including existing methods based on thresholding, region growing, boundary identification, and stochastic modeling. However, these existing methods suffer from other limitations, such as requiring user inputs, inaccurate performance when model assumptions are not satisfied, and sensitivity to the PVEs. Learning-based methods have been developed to address these issues, but existing learning-based methods require manual segmentations for training, which are likely affected by the PVEs. A recently developed deep-learning (DL)-based technique accounts for the PVEs arising due to system blur, but does not model the TFEs due to implementing segmentation as a pixel-wise classification task. Most often, existing methods exclusively assign each pixel as belonging to either the tumor or the normal tissue class and thus, do not provide for a voxel including a mixture of both tumor and normal tissues. Fuzzy segmentation methods have attempted to account for the TFEs by assigning a finite number of “fuzzy levels” for pixels containing mixed tissue classes, but the finite “fuzzy levels” in these methods allow only for a discrete representation of the tumor-fraction area within each pixel and does not provide for modeling tumor-fraction areas as continuous random variables. Therefore, existing fuzzy segmentation methods are still considered to be classification-based methods. To accurately model the TFEs, it is desired to yield a continuous estimate of the tumor-fraction area within each pixel.

To address these issues, the development and assessment of an estimation-based segmentation method is disclosed below that accounts for both sources of the PVEs described above. We develop a Bayesian estimator that estimates the posterior mean of the tumor-fraction area within each pixel. Further, through a physics-guided learning process, the estimator accounts for the blur arising due to the limited spatial resolution. The method is implemented using an auto-encoder. As described below, the disclosed method is quantitatively evaluated in the context of segmenting primary tumors in 18F-fluorodeoxyglucose (FDG)-PET images of patients with non-small cell lung cancer, the most common lung cancer subtype with high mortality rates. The results of these experiments demonstrated that the disclosed method yielded reliable estimates of the tumor-fraction area within each pixel, outperformed conventional segmentation methods, and was relatively insensitive to both sources of the PVEs.

### Methods

### Theory

Consider a PET system imaging a tracer distribution, described by a vector ƒ(r), where r denotes the spatial coordinates. In this manuscript, we consider a 2D slice-by-slice imaging, so that r=(x, y). The function ƒ(r) describes both the tumor and the rest of the regions with tracer uptake. We denote the tracer uptake in the tumor by ƒₛ(r). The rest of the regions are referred to as background, and uptake in the background is denoted as ƒ_{b}(r). Thus the tracer uptake can be represented mathematically as follows:

ƒ(r)=ƒ_{b}(r)+ƒₛ(r) Eqn. (16)

We define a support function for the tumor region as s(r), i.e.

$$s ( r ) = \{ \begin{matrix}1 , & if {f}_{s} ( r ) > 0. \\ 0 , & otherwise\end{matrix} . \qquad Eqn . ( 17 )$$

The tracer emits photons that are detected by the PET imaging system to yield projection data. The projection data is then used to reconstruct the image over an M-dimensional pixelated grid. Denote the pixelated reconstructed image by an M-dimensional vector \hat{f}. Thus, the mapping from the tracer distribution to the reconstructed image is given by the operator Θ: [symbol] ₂( [symbol] ²)→ [symbol] ^{M}.

Denote the PET system by a linear continuous-to-discrete operator [symbol] and let the vector n describe the photon noise. Denote the reconstruction operator, quite possibly non-linear, by [symbol] . The eventual reconstructed image is given in operator notation as below:

\hat{f}= [symbol] { [symbol] f+n} Eqn. (18)

In the reconstructed image, denote the pixel width by L, and the x and y coordinates of the center of the m^{th} pixel by xₘ and yₘ, respectively. Then the pixel basis is given by:

$${ϕ}_{m} ( r ) = \frac{1}{{L}^{2}} rect ( \frac{x - {x}_{m}}{L} ) rect ( \frac{y - {y}_{m}}{L} ) \qquad Eqn . ( 19 )$$

The fractional area that the tumor occupies in the m^{th} pixel, denoted by aₘ, is given by

$${a}_{m} = \frac{1}{{L}^{2}} \int s ( r ) {ϕ}_{m} ( r ) {d}^{2} r \qquad Eqn . ( 20 )$$

Our objective is to design a method that estimates this quantity am from the reconstructed image \hat{f} for all M pixels. Denote the estimate of aₘ by âₘ. Further, denote the M-dimensional vector {a₁, a₂, . . . , aₘ} by a, and denote the estimate of a by â.

Estimating â from the reconstructed image is an ill-posed problem due to the null spaces of the [symbol] and [symbol] operator. Thus, to estimate â, we take a Bayesian approach. We first need to define a cost function that penalizes deviation of a from â. A common cost function is the ensemble mean squared error (EMSE), which is the mean squared error averaged over noise realizations and the true values a. However, in our case, the variable âₘ is constrained to lie between [0, 1]. The EMSE loss does not directly incorporate this constraint. In contrast, using the binary cross-entropy (BCE) loss as the penalizer allows us to incorporate this constraint on âₘ directly. The BCE loss between aₘ and âₘ, denoted by l_{BCE}(aₘ, âₘ) is given by

l_{BCE}(aₘ,âₘ)=aₘ log(âₘ)+(1−aₘ)log(1−âₘ) Eqn. (21)

We define our cost function C(a,â) as the aggregate BCE loss over all pixels averaged over the joint distribution of a and the noise realization \hat{f}. Denote [symbol] . . . [symbol] X as the expected value of the quantity in the brackets when averaged over the random variable X Thus, the cost function is defined as:

$$\begin{matrix}C ( a , \hat{a} ) = 〈 {\sum }_{m = 1}^{M} \text{?} ( {a}_{m} , {\hat{a}}_{m} ) 〉 \text{?} \\ = 〈 {〈 {\sum }_{m = 1}^{M} \text{?} ( {a}_{m} , {\hat{a}}_{m} ) 〉}_{a \vert \hat{f}} 〉 \text{?} \\ = \int {d}^{M} \text{?} pr ( \text{?} ) \int {d}^{M} a pr ( a \vert \hat{f} ) {\sum }_{m = 1}^{M} [ {a}_{m} log ( {\hat{a}}_{m} ) + \\ ( 1 - {a}_{m} ) log ( 1 - {\hat{a}}_{m} ) ] .\end{matrix} \qquad Eqn . ( 22 )$$

$$\text{?} \text{indicates text missing or illegible when filed}$$

To estimate the point at which this cost function is minimized, we differentiate the cost function with respect to the vector â and set that equal to zero. Because pr(\hat{f}) is always non-negative, the cost function is minimized by setting

$$0 = \frac{\partial }{\partial \hat{a}} 〈 {\sum }_{m = 1}^{M} [ {a}_{m} log ( \text{?} ) + ( 1 - {a}_{m} ) log ( 1 - {\hat{a}}_{m} ) ] 〉 \text{?} \\ \text{?} \text{indicates text missing or illegible when filed} \qquad Eqn . ( 23 )$$

This is then equivalent to performing component-wise differentiation and setting each differentiated component to 0. Conducting this operation for the m^{th} component yields

$$0 = \frac{\partial }{\partial {\hat{a}}_{m}} 〈 {a}_{m} [ log ( \text{?} ) - log ( 1 - {\hat{a}}_{m} ) ] 〉 \text{?} + \frac{\partial }{\partial {\hat{a}}_{m}} 〈 log ( 1 - {\hat{a}}_{m} ) 〉 \text{?} . \qquad Eqn . ( 24 )$$

$$\text{?} \text{indicates text missing or illegible when filed}$$

Define the solution to the above equation by âₘ* Eqn. (24) yields

$${\hat{a}}_{m}^{*} = {〈 {a}_{m} 〉}_{{a}_{m} \vert f} \text{?} \qquad Eqn . ( 25 )$$

$$\text{?} \text{indicates text missing or illegible when filed}$$

or equivalently, in vector notation as

a*= [symbol] a [symbol] _{a|\hat{f}} Eqn. (26)

Therefore, minimizing the cost function in Eqn. (22) results in the optimal estimator equivalent to the posterior mean. Note that the same estimator is yielded when the cost function is the EMSE. We can further show that this estimator is unbiased in a Bayesian sense:

â̅*̅=∫d^{M}\hat{f}pr(\hat{f})∫d^{M}apr(a|\hat{f})a

=∫d^{M}apr(a)a=ā. Eqn. (27)

Thus, we show that by minimizing the cost function defined in Eqn. (22), we can get a posterior mean estimate of the tumor-fraction area in each pixel of the reconstructed image. This estimator will be unbiased in a Bayesian sense. From the estimated tumor-fraction area values, we can define a segmented map of the tumor using a procedure described below.

### Implementation of the Estimator

Estimating the posterior mean â* requires sampling from the posterior distribution pr(a|\hat{f}). Sampling from this distribution is challenging as this distribution is high-dimensional and does not have a known analytical form. To address this issue, we constructed an autoencoder that minimized the cost function given by Eqn. (22). In this autoencoder, each intermediate layer was normalized using batch normalization to stabilize the learning process. Skip connections with element-wise addition, as implemented in a recently developed U-net-based segmentation method, were applied to feed the features extracted in the down-sampling path into the up-sampling path to improve the learning performance. The autoencoder was trained via the Adam optimization algorithm. In the various experiments mentioned below, this autoencoder was optimized with a training set using five-fold cross-validation.

### Evaluation

Evaluating the disclosed method requires access to ground truth where the true tumor-fraction area map or a surrogate for this true tumor-fraction area map is known. For this purpose, we conducted realistic simulation studies. In these studies, the tumor can be described at a very high resolution, i.e. ƒₛ(r) in Eqn. (16), thus providing a rigorous framework to evaluate the method. From this high-resolution description, the fractional area of each pixel containing the tumor region can be computed using Eqn. (20). However, using simulations to evaluate the disclosed segmentation method would require that the simulated images themselves are highly realistic. We thus first describe the strategy to conduct the realistic simulation studies and validate the realism of the images.

### Simulation Framework

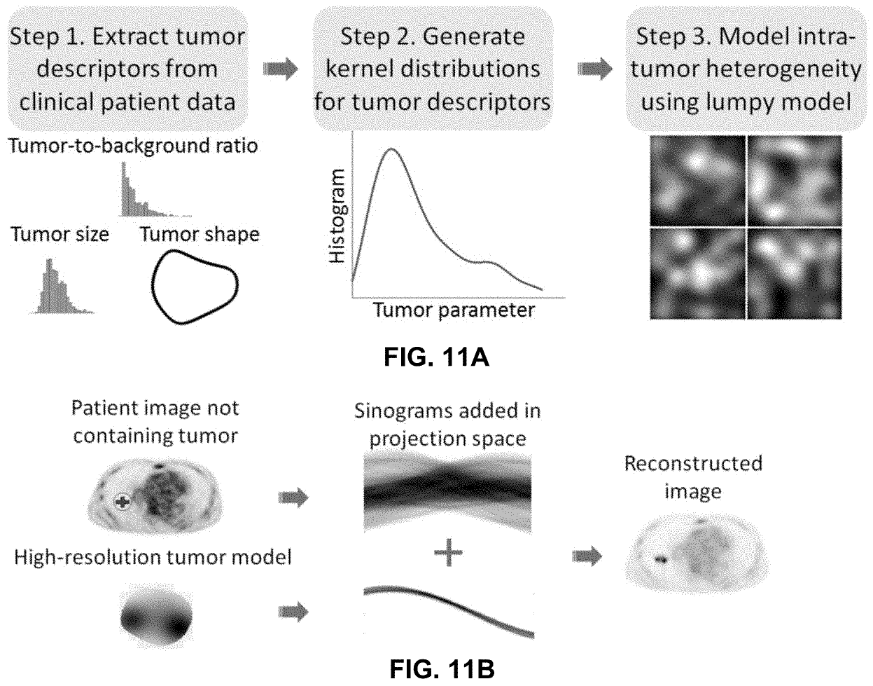

In the first step, a realistic high-resolution tumor model was developed to capture the observed variabilities in tumor properties from an actual patient population (FIG. 11A). For this purpose, we extended upon a previously-published simulation framework. Summarizing, first, tumor descriptors including shape, size and tumor-to-background intensity ratio were extracted from PET images of patients with non-small cell lung cancer. Tumor shape was quantified by five harmonic elliptical Fourier descriptors. Tumor size was quantified by diameter. The distribution of each tumor descriptor was modeled using a stochastic kernel density estimation approach. The kernel distribution of each descriptor was then sampled, and from the sampled parameters, simulated tumors were generated. Necrosis within the tumor was modeled in the previously-published model by assigning a lower intensity to the tumor core compared to the rim core. We extended that framework to model the intra-tumor heterogeneity more generally. We used the observation that the tracer uptake patterns within tumors can be modeled as a combination of lumps, where the lump locations, amplitudes, and sizes are random variables. More generally, it is suitable to characterize the tracer uptake within a tumor as a random process. Thus, the intra-tumor heterogeneity was modeled through a stochastic lumpy object model. This lumpy model was inspired by a previously-published lumpy background model, with some adaptations to model intra-tumor heterogeneity. The lumpy model is defined as

$$\begin{matrix}f ( r ) = s ( r ) {\sum }_{n = 1}^{N} Λ ( r - {c}_{n} \vert {a}_{n} , {σ}_{n} ) \\ = s ( r ) {\sum }_{n = 1}^{N} \frac{{a}_{n}}{a {πσ}_{n}^{2}} exp ( - \frac{{\vert r - {c}_{n} \vert}^{2}}{{σ}_{n}^{2}} )\end{matrix} \qquad Eqn . ( 28 )$$

where s(r) denotes the support for the tumor, N denotes the total number of lumps, A(⋅) denotes the lump function, r denotes the spatial coordinate in two dimensions, cₙ, aₙ and σₙ denote the center, magnitude and width of the n^{th} lump function, respectively.

To model the tracer uptake as a random process, cₙ was uniformly distributed within the defined tumor support, and aₙ and σₙ were uniformly distributed within a pre-defined range but appropriately scaled based on the clinically extracted values of the tumor-to-background intensity ratios. All these parameters were used to generate the tumor models.

Since the ground truth was not needed for the background, existing patient images containing lung cavity but with no tumor present were selected as templates for the background. This was done to ensure that the background was realistic.

In the second step (FIG. 11B), realistic forward projections for the tumor and background were generated by simulating a PET imaging system. The high-resolution tumor image and the low-resolution patient image were passed through corresponding PET system models to obtain sinograms with clinically relevant count levels. The simulation modeled the major image-degrading processes of system blur and photon noise. The sinograms were reconstructed using a 2D ordered subset expectation maximization (OSEM) algorithm. Adding the data in the projection space and then performing the reconstruction helped incorporate the impact of noise texture on the tumor appearance in the reconstructed image. The realism of the tumors in these simulated PET images was evaluated as described below.

### Evaluating the Realism of the Tumors in the Simulated PET Images

To evaluate the realism of the tumors, we followed an approach similar to previously-published studies on evaluating lesion-insertion techniques for CT images. Our objective was to quantitatively evaluate whether trained readers could distinguish between real and simulated tumors. For this purpose, we conducted a human observer study using a two-alternative-forced-choice (2-AFC) protocol.

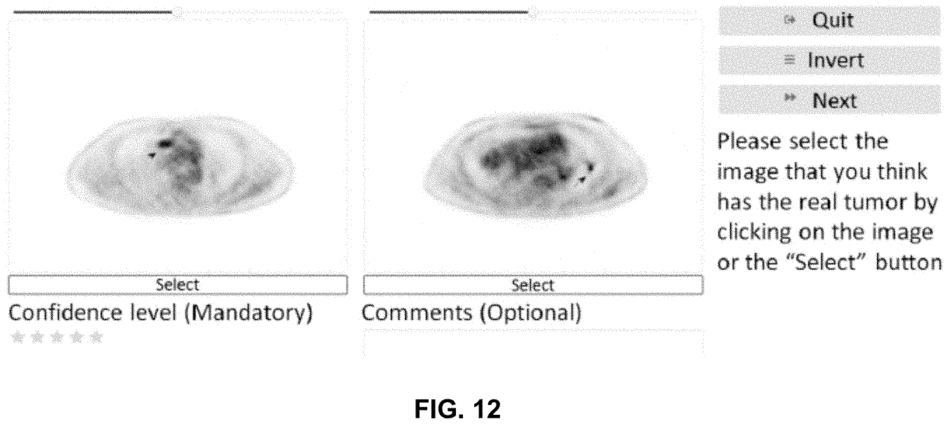

We developed a web-based app (FIG. 12) where trained readers were shown two images, one a real patient image containing a real tumor, and the other a simulated image generated using our simulation framework. In the app, readers were instructed to identify the tumor they thought was real. The tumor locations were shown in the images to ensure that the readers were focusing on the tumor-realism task, and not treating this as a tumor-detection task. Additionally, the app provided interfaces to invert the tumor intensities and adjust the tumor contrast, to facilitate a rigorous evaluation. Readers identified which image contained the real tumor and provided a confidence level for their decisions. The confidence levels were interpreted as summarized in Table 1 below.

TABLE I
Definition of the confidence scale for the 2-AFC study

| Confidence Level | Definition |
|---|---|
| 1 | No confidence in determination |
| 2 | Possibly correct |
| 3 | Probably correct |
| 4 | Likely correct |
| 5 | 100% confidence in determination |

Six trained readers (five board-certified radiologists with specialization in nuclear medicine and years of experience in reading PET images, and one experienced nuclear-medicine physicist performed the app-based evaluation for 50 image pairs. We computed the fraction of times that each reader correctly identified the image containing the real tumor. A percent accuracy close to 50% indicated that the simulated images were highly realistic.

This simulation framework was used for evaluating the disclosed segmentation method as described below.

### Quantitative Evaluation of the Disclosed Segmentation Method

The disclosed estimation-based segmentation method was quantitatively evaluated to evaluate the accuracy of the disclosed method, to evaluate the sensitivity of the disclosed method to PVEs, to compare the disclosed method to existing techniques, and to evaluate the performance of the disclosed method with different clinical scanner configurations. For each of the experiments, we generated PET images with tumors using the simulation framework described above. The generated dataset was split into training and test sets. The autoencoder was trained and validated (five-fold cross-validation) using the training dataset. The performance of the autoencoder was then evaluated using a completely independent test data. For each of the experiments below, we mention the number of training and test images that were used individually.

Since the disclosed method is an estimation-based segmentation approach, the disclosed method was evaluated using metrics to evaluate performance on estimation tasks and segmentation tasks.

Overall performance on the estimation task was evaluated using the EMSE metric. This metric provides a combined measure of bias and variance over the distribution of true values and noise realizations and thus is a comprehensive figure of merit for estimation tasks. The pixel-wise EMSE denotes the mean squared error between the true and estimated tumor-fraction area vectors, averaged over noise realizations and all true values of the tumor-fraction area. Mathematically:

Pixel-wise EMSE= [symbol] ∥â−a∥₂² [symbol] _{\hat{f}|a} [symbol] ₐ Eqn. (29)

The area EMSE denotes the EMSE between the true and estimated areas of each tumor. The true and estimated areas, denoted by A and Â, are simply given by the L₁ norms of a and â, respectively. The Area EMSE is then given by:

Area EMSE= [symbol] |Â−A|² [symbol] _{\hat{f}|A} [symbol] _{A} Eqn. (30)

In Eqn. (15), we showed that the disclosed method should yield an estimate of the tumor-fraction area that is unbiased in a Bayesian sense. To check for this, the ensemble-average bias was computed. This term, denoted by b, is an M-dimensional vector {b₁, b₂, . . . , b_{M}}, with the m^{th} element of the vector quantifying the average bias of the estimated tumor area in the m^{th} pixel. Consider a total of P tumor images and N noise realizations for each tumor image. Let aₘₙₚ and âₘₙₚ denote the true and estimated tumor-fraction area within the m^{th} pixel for the n^{th} noise realization, in the p^{th} tumor image. The m^{th} ensemble-average bias term b̅ₘ is then given by

$${\overline{b}}_{m} = \frac{1}{P} {\sum }_{p = 1}^{P} \frac{1}{N} {\sum }_{n = 1}^{N} [ {\hat{a}}_{mnp} - {a}_{mnp} ] \qquad Eqn . ( 31 )$$

The proximity of the b̅ to 0 indicates the estimator being unbiased in a Bayesian sense.

Evaluation on the segmentation task was conducted using spatial-overlap-based, volume-based, and spatial-distance-based metrics. The spatial-overlap-based and volume-based metrics used in this study are derived based on the four cardinalities of the confusion matrix: true positives (TP), the false positives (FP), the true negatives (TN) and the false negatives (FN). Note that the four cardinalities are generalized to evaluate segmentation methods that assign a continuous “fuzzy level” to each pixel, and thus are suitable for the evaluation of the disclosed method. The four cardinalities are described as

$$TP = {\sum }_{m = 1}^{M} min ( {\hat{a}}_{m} , {a}_{m} ) , \\ FP = {\sum }_{m = 1}^{M} max ( {\hat{a}}_{m} - {a}_{m} , 0 ) , \\ TN = {\sum }_{m = 1}^{M} min ( 1 - {\hat{a}}_{m} , 1 - {a}_{m} ) , \\ FN = {\sum }_{m = 1}^{M} max ( ( 1 - {\hat{a}}_{m} ) - ( 1 - {a}_{m} ) , 0 ) \qquad Eqn . ( 32 )$$

The metric of fuzzy Dice Similarity Coefficient (fDSC) and fuzzy Jaccard Similarity Coefficient (fJSC) were used to measure the spatial-overlap agreement between the ground-truth and estimated segmentation. The fDSC and fJSC are defined as

$$fDSC = \frac{2 TP}{2 TP + FP + FN} \qquad Eqn . ( 33 )$$

$$fJSC = \frac{TP}{TP + FP + FN} \qquad Eqn . ( 34 )$$

Higher values of fDSC and fJSC indicate higher segmentation accuracy. The metric of Volume Similarity (VS) was used to measure the similarity between the true and measured volumes:

$$VS = 1 - \frac{\vert FN - FP \vert}{2 TP + FP + FN} \qquad Eqn . ( 35 )$$

Higher values of VS indicate higher accuracies in metabolic tumor volume measurement. In this case, since we were investigating the segmentation performance in 2D images, VS implied the area similarity. Finally, to assess the similarity in the true boundary and the boundary generated by the segmentation technique, the Hausdorff Distance (HD) was used. For computing the HD, we only considered those cases where the tumor had been correctly localized by the disclosed segmentation method. For these cases, using the estimated values of tumor-fraction areas, a tumor topographic map was defined. From this topographic map, the set of points at which the value of the tumor-fraction area was 0.5 was defined as the tumor contour. The coordinates of points along these contour lines were used to define the tumor boundary for the HD calculation:

$$HD = max ( {max}_{c ∈ C} {min}_{\hat{c} ∈ \hat{C}} ‖ c - \hat{c} ‖ , {max}_{\hat{c} ∈ \hat{C}} {min}_{c ∈ C} ‖ \hat{c} - c ‖ ) \qquad Eqn . ( 36 )$$

where c of the set C and ĉ of the set Ĉ refer to the coordinate of the point along the true and estimated tumor boundary, respectively. Lower values of HD indicate higher accuracy in tumor-boundary delineation.

We also qualitatively assessed the performance of the disclosed method on the boundary-delineation task. The boundary as defined above was compared to the known continuous ground truth from the high-resolution tumor image. The boundary was also compared to the boundary obtained with a recently developed U-net-based PET segmentation technique. This U-net-based method, like most conventional segmentation methods, exclusively classifies each pixel as either tumor or background.

All segmentation metrics were reported as mean (95% confidence intervals).

The disclosed method was quantitatively compared to various commonly used semi-automated PET segmentation methods, including region growing with 40% SUV-max thresholding method, active-contour-based snakes method, and a Markov random fields-Gaussian mixture model (MRF-GMM) technique. The disclosed method was also compared to the fuzzy local information C-Means clustering algorithm (FLICM). Additionally, the method was compared to the U-net-based method described above. For all the semi-automated segmentation methods, a region of interest (ROI) containing the tumor was provided as manual input. The disclosed method and the DL-based segmentation method did not require any user input.

We acquired 250 2-D slices from 15 patients for the background portion of the image in the simulation framework. The simulated PET imaging system was modeled with a 5 mm FWHM system blur. The sinograms were reconstructed using the OSEM algorithm with 21 subsets and 2 iterations, which were selected to be similar to the PET reconstruction protocol for the patient images. The reconstructed pixel size was 4.07 mm×4.07 mm. The autoencoder was trained and cross-validated using 6400 and 1800 simulated images, respectively. Evaluation was then performed on 1800 completely independent simulated images.

To evaluate the sensitivity of the disclosed method to PVEs, we studied the performance of the method as a function of tumor sizes. For this purpose, all test images were grouped based on the range of the true tumor sizes. For each test image, PVE-affected tumor boundaries were generated by applying a rectangular filter to the ground-truth tumor mask. This filter modeled the resolution degradation due to the system blur and the reconstruction process. The filter size was equal to the FWHM of the Gaussian blur in a reconstructed image, which was generated by imaging a line phantom using the PET system.

The PVE-affected tumor area and the tumor area measured using the disclosed method were obtained. Each of these results was divided by the true tumor area. A ratio of 100% indicated that the output was insensitive to PVEs. A ratio lower or higher than 100% indicated an underestimation or overestimation of the tumor area, respectively, showing that the segmentation output was affected by the PVEs. For computing the ratios, only cases with correct tumor localization were considered.

To study the performance of the disclosed method in a highly clinically realistic setting, we simulated two PET imaging systems based on Siemens Biograph 40 and Biograph Vision. The process to generate PET images requires availability of patient images with no tumors. For each of these scanners, we obtained clinical PET images. These PET images had different voxel sizes as dictated by the imaging protocol. The Biograph 40 generated images of 128×128 pixels, while the Biograph Vision generated images of 192×192 pixels. Details on the PET scanner acquisition and reconstruction parameters are summarized in Table 2. Using these patient images, a total of 6240 simulated 2D PET images were generated for each scanner. Following the training and optimization, the disclosed method was tested on 1560 newly simulated images. For comparison, the U-net-based method was also evaluated.

TABLE 2
Technical acquisition and reconstruction
parameters of the PET systems.

| | Siemens | Siemens |
|---|---|---|
| | Biograph | Biograph |
| Parameters | 40 | Vision |
| Transaxial FOV (mm) | 550 | 700 |
| Axial FOV (mm) | 216 | 162 |
| Reconstruction method | OSEM | OSEM |
| Subsets | 21 | 21 |
| Iterations | 2 | 2 |
| Crystal pitch (mm) | 4.00 | 3.30 |
| FWHM (mm) @ 1 cm | 5.90 | 3.70 |
| Voxel size (mm3) | 4.30 | 3.65 |

### Results

### Evaluating the Realism of the Tumors in the Simulated PET Images

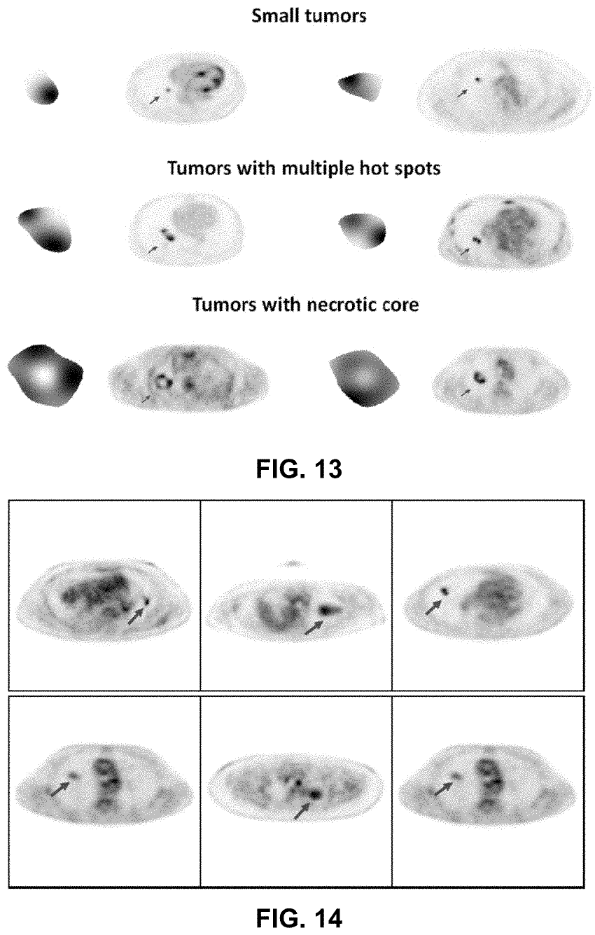

Representative simulated images obtained using the disclosed simulation framework are shown in FIG. 13. These images demonstrate the capability of the strategy to generate a wide variety of clinically observed tumor types such as those with small size (top tow), those with multiple hot spots (middle row), and those with necrotic cores (bottom row).

The results evaluating the realism of the simulated images using the 2-AFC study described above are summarized in Table 3. Each trained reader could identify the tumor accurately only approximately 50% of the time, indicating that the simulated images were highly realistic. In addition, all the mean confidence levels were lower than 4, indicating that the users were not highly confident about their decisions.

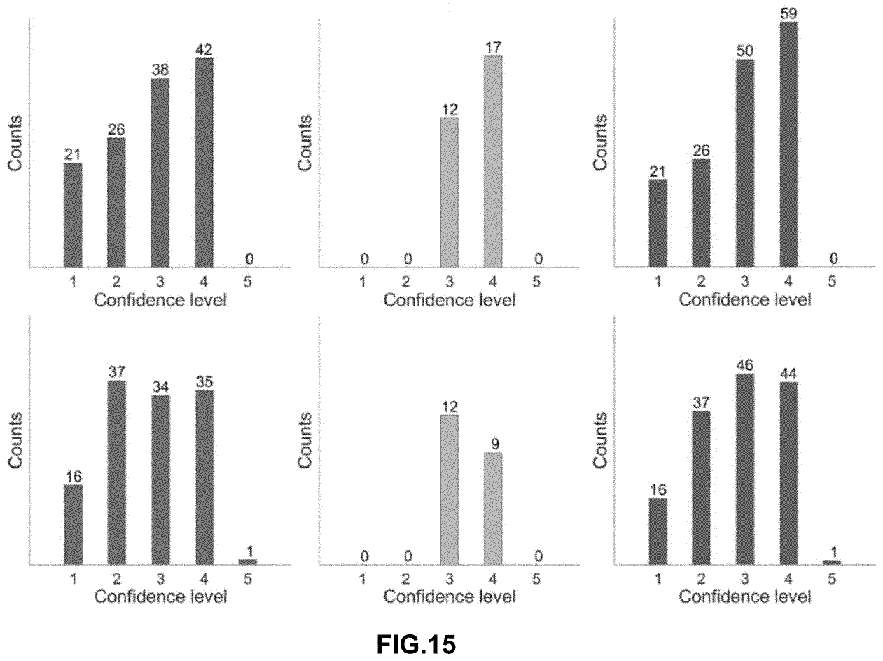

FIG. 15 shows the distribution of the confidence levels for two reader groups and all readers included when correctly (upper row) and incorrectly (lower row) identified the image pairs. The five board-certified radiologists with years of experience in reading PET images correctly identified 127/250 (51%) image pairs in total. Among the 127 correct identifications, only 42 (33%) of them were selected with confidence levels≥4. This indicates that the readers were not highly confident even when they made correct identifications. Examples of the simulated images for which at least half of the readers incorrectly identified the tumor with high confidence levels (confidence levels≥3) are shown in FIG. 14. This result indicates that the readers were incorrectly identifying the tumors even when they were confident about their decisions. This was true over a range of tumor types, demonstrating further the robustness of the simulation framework. Overall, these results demonstrate that the simulated images were highly realistic.

### Evaluating the Performance of the Disclosed Segmentation Method and Comparing to Other Segmentation Methods

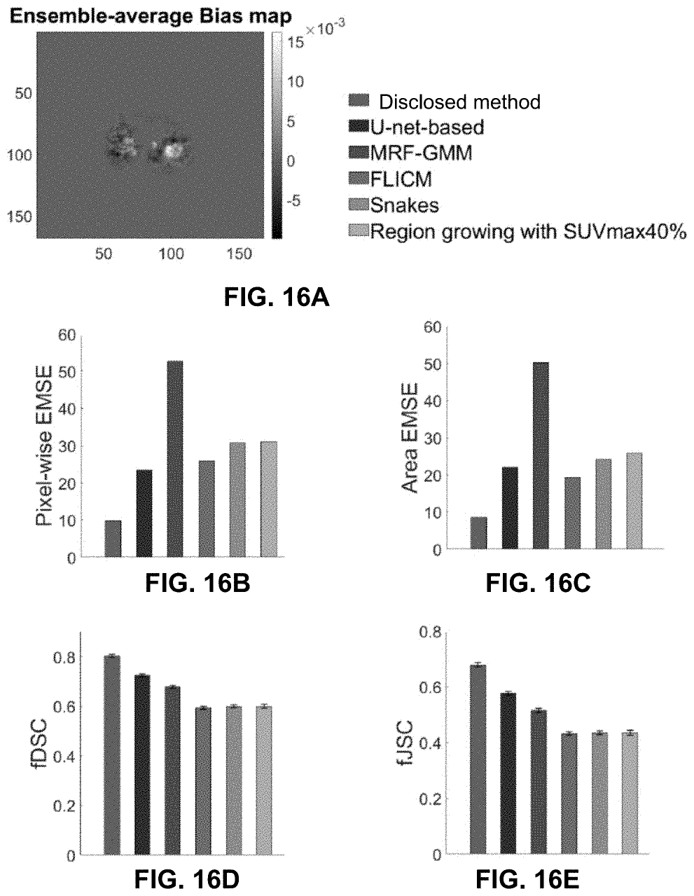

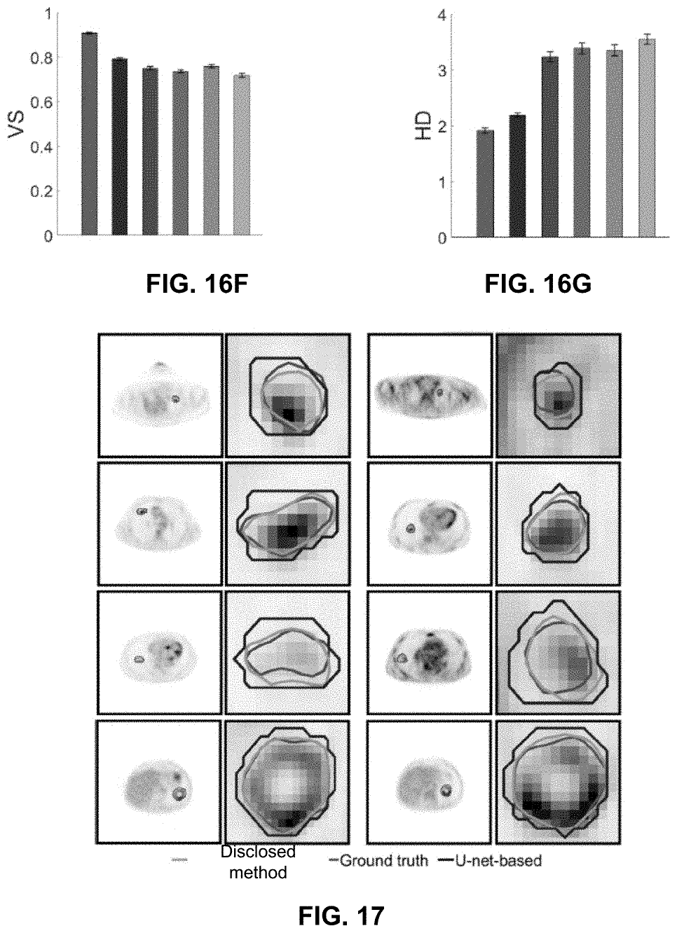

Quantitatively, the disclosed method significantly outperformed (p<0.01) the other considered methods, including the U-net-based PET segmentation method, on the basis of pixel-wise EMSE, area EMSE, fDSC, fJSC, VS, and HD metrics (FIGS. 16A, 16B, 16C, 16D, 16E, 16F, 16G and Table 4). The disclosed method yielded the lowest pixel-wise EMSE, the lowest area EMSE, the highest fDSC of 0.80 (95% CI: 0.80, 0.81), fJSC of 0.68 (95% CI: 0.67, 0.69) and VS of 0.91 (95% CI: 0.90, 0.91), and the lowest HD of 1.92 (95% CI: 1.87, 1.96) (Table 4). In addition, the ensemble-average bias map yielded all values close to 0, demonstrating the capability of the estimator to yield an unbiased estimate of the tumor-fraction area within each pixel.

TABLE 3
Percent accuracy and mean confidence level for each
trained reader participating in the 2-AFC study.

| | | Mean |
|---|---|---|
| | Percent | Confidence |
| | Accuracy | Level |
| NM Physician 1 | 44% | 1.76 |
| NM Physician 2 | 50% | 7.58 |
| NM Physician 3 | 58% | 3.14 |
| NM Physician 4 | 58% | 3.80 |
| NM Physician 5 | 44% | 3.54 |
| NM Physicist 1 | 58% | 3.52 |

NM denotes nuclear medicine.

TABLE 4
Comparison between the disclosed method and other methods
on simulated images using quantitative figures of merit.

| | Pixel- | | | | | |
|---|---|---|---|---|---|---|
| | wise | Area | | | | |
| | EMSE | EMSE | fDSC | fJSC | VS | HD |
| Disclosed | 9.89 | 8.56 | 0.80 | 0.68 | 0.91 | 1.92 |
| Method | | | (0.80, 0.810) | (0.67, 0.69) | (0.90, 0.91) | (1.87, 1.96) |
| U-net- | 23.31 | 22.04 | 0.72 | 0.58 | 0.79 | 2.19 |
| based | | | (0.72, 0.73) | (0.57, 0.58) | (0.79, 0.80) | (2.16, 2.23) |
| MRF- | 52.74 | 50.13 | 0.68 | 0.52 | 0.75 | 3.24 |
| GMM | | | (0.67, 0.69) | (0.51, 0.52) | (0.74, 0.76) | (3.15, 3.33) |
| FLICM | 25.97 | 19.39 | 0.59 | 0.43 | 0.74 | 3.39 |
| | | | (0.59, 0.60) | (0.43, 0.44) | (0.73, 0.74) | (3.29, 3.48) |
| Region | 31.11 | 25.78 | 0.60 | 0.43 | 0.76 | 3.35 |
| growing | | | (0.59, 0.61) | (0.43, 0.44) | (0.75, 0.77) | (3.26, 3.45) |
| with 40% | | | | | | |
| SUVₘₐₓ | | | | | | |
| Snakes | 30.73 | 24.14 | 0.60 | 0.44 | 0.72 | 3.55 |
| | | | (0.59, 0.61) | (0.43, 0.44) | (0.71, 0.73) | (3.46, 3.64) |

Values are reported as mean (95% confidence interval).

Qualitatively, FIG. 17 shows the comparison between the ground-truth tumor boundaries and the boundaries yielded by the disclosed method. Results with the U-net-based method are also shown for comparison. These results demonstrate that the disclosed method provides a better match to the high-resolution ground-truth contours for different tumor types, including tumors with complicated heterogeneity patterns.

### Evaluating the Sensitivity of the Disclosed Method to the PVEs

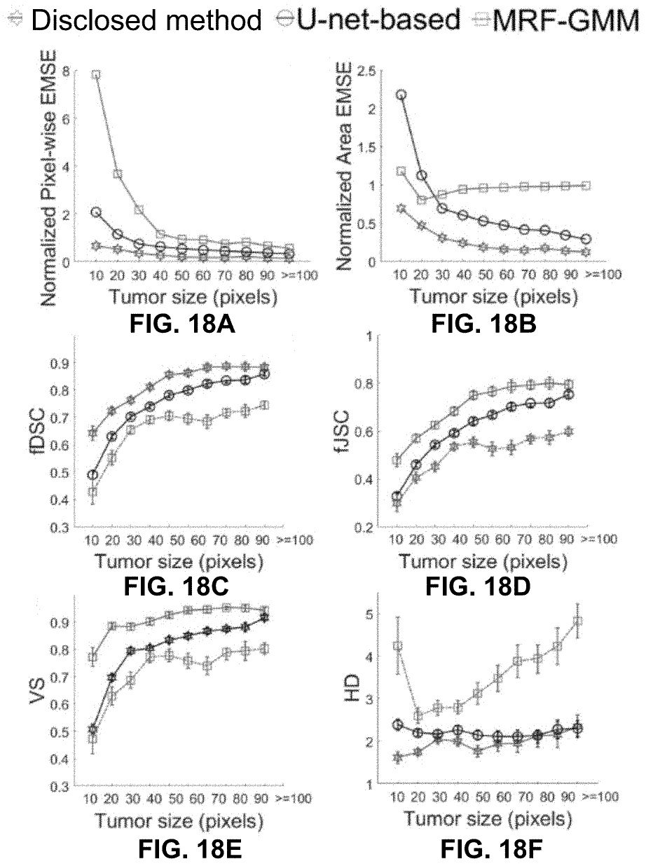

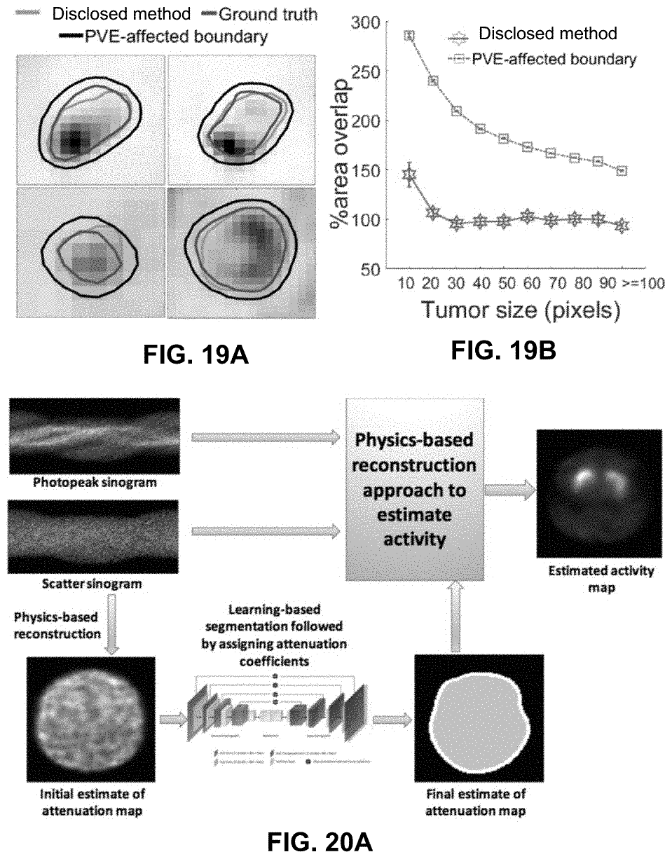

FIGS. 18A and 18B shows that the disclosed method consistently yielded lowest normalized pixel-wise and area EMSE, where both the normalizations were performed over the true tumor area for each test image. In addition, the disclosed method yielded the highest fDSC, fJSC and VS, and the lowest HD for all tumor sizes (see FIGS. 18C, 18D, 18E, and 18F, respectively). FIGS. 19A and 19B show that the method consistently yielded better estimate of tumor area compared to the PVE-affected tumor boundaries for all tumor sizes, thus demonstrating the capability of the method to account for PVEs. In addition, the disclosed method significantly outperformed (p<0.01) the U-net-based method and was less sensitive to the PVEs.

Evaluating the Disclosed Method with Different Clinical Configurations

Table 5 shows the comparison of the segmentation accuracy between the disclosed method and the U-net-based method for the two scanner configurations described above. The disclosed method significantly outperformed (p<0.01) the U-net-based method for both the configurations, on the basis of fDSC, fJSC, VS and HD metrics. In addition, the segmentation accuracy is higher for the Biograph Vision configuration, which has a higher scanner resolution and lower voxel size compared to the Biograph 40 (Table 2).

TABLE 5
Comparison between the disclosed method and the
U-net-based method on clinical relevance study.

| | 128 × 128 | | 192 × 192 | |
|---|---|---|---|---|
| | Disclosed | U-net-based | Disclosed | U-net-based |
| | method | method | method | method |
| fDSC | 0.816 | 0.708 | 0.854 | 0.742 |
| | (0.811 0.821) | (0.703 0.713) | (0.850 0.858) | (0.737 0.746) |
| fJSC | 0.694 | 0.551 | 0.744 | 0.593 |
| | (0.688 0.701) | (0.545 0.558) | (0.738 0.750) | (0.587 0.599) |
| VS | 0.918 | 0.746 | 0.936 | 0.771 |
| | (0.915 0.921) | (0.740 0.752) | (0.933 0.939) | (0.766 0.775) |
| HD | 1.744 | 2.290 | 1.752 | 2.340 |
| | (1.712 1.775) | (2.260 2.320) | (1.719 1.785) | (2.304 2.377) |

### Discussion

In this manuscript, we evaluated the method through simulation studies, where the high-resolution ground-truth tumor information can be defined. For clinical relevance, we conducted a 2-AFC study to evaluate the realism of the simulated tumors. Results from FIG. 13 demonstrated the capability of our simulation framework to model high variabilities of clinically realistic tumor types, including necrotic tumors. The results from the 2-AFC study, as shown in FIG. 15 and Table 3, provided strong evidence of the realism of the simulated tumors. These results further demonstrated the capability of the disclosed stochastic lumpy object model to characterize clinically realistic tumor uptake patterns, including tumor necrosis.

Qualitatively, the method demonstrated its capability to accurately estimate the high-resolution ground-truth tumor boundaries. FIG. 7 shows that the disclosed method was able to yield non-pixelated tumor contours and a better match to the ground-truth contour, because of the capability to yield continuous estimates of the tumor-fraction areas. Note that for pixel-wise classification-based segmentation methods such as the U-net-based method, the estimated contours are highly pixelated and thus can lead to severe overestimation for smaller tumors. In addition, the method was capable to yield accurate performance in complicated PET segmentation tasks, where complex heterogeneity patterns can reside in highly heterogeneous backgrounds (i.e., uptakes in medistinum and lymph nodes).

From the evaluation studies, we observed that the method yielded more accurate delineations of tumor boundaries by learning the high-resolution ground-truth tumor-fraction area information during training. This indicates that when the access to the tumor definition at a higher resolution is available, the method can be trained to perform a more accurate tumor-segmentation task in poorer resolution images. This observation has motivated us to further evaluate our method in physical phantom studies, where the high-resolution tumor definitions can be obtained from CT images. To evaluate the performance of the method in real patient images, the method can be trained on patient images obtained from the lower resolution PET scanner along with the ground-truth tumor definition retrieved from a higher-resolution PET scanner. This would require the same patient to undergo both scanners. This can be potentially addressed by applying virtual-pinhole PET insert technology to obtain such high-resolution tumor boundary information without interfering with the clinical scanning protocol. However, this would require sufficient amount of patient images to be collected for training purposes. Another potential strategy to apply the method in real patient images is to pre-train the method on either digital simulation or physical phantom studies. The method can then be fine-tuned on real patient images.

### Conclusion

In this example, we disclosed an estimation-based PET segmentation method that yields the posterior mean estimate of the tumor-fraction area for each pixel, which is then used to define the high-resolution segmented tumor boundaries. The method was demonstrated to yield reliable estimates of the tumor-fraction areas. In addition, the method yielded superior tumor-delineation performance, was relatively insensitive to PVEs, and significantly outperformed commonly used PET segmentation methods, including an U-net-based method, in trained-readers-validated simulated 2D PET images. In conclusion, the disclosed method yields accurate tumor delineations and accounts for both sources of PVEs.

### Example 3: Transmission-Less Attenuation Compensation Method for Brain SPECT Imaging

The following example describes a method of estimating an attenuation distribution using information contained within scattered photons in SPECT imaging. A physics-based and learning-based method that uses the SPECT emission data in the photopeak and scatter windows to perform transmission-less attenuation and scattering compensation (ASC) in SPECT imaging. The disclosed method was developed in the context of quantitative 2-D dopamine-transporter (DaT)-scan SPECT imaging.

The disclosed method makes use of data acquired in the scatter window to reconstruct an initial estimate of the attenuation map using a physics-based approach. An autoencoder is then trained to segment this initial estimate into soft tissue and bone regions. Pre-defined attenuation coefficients are assigned to these segmented regions, yielding a reconstructed attenuation map. This attenuation map is used to reconstruct the activity distribution using an ordered subsets expectation maximization (OSEM)-based reconstruction approach.

We objectively evaluated the performance of this method using highly realistic simulation studies, where the task was to quantify activity uptake in the caudate and putamen regions of the brain from the reconstructed activity images. The results of this evaluation indicated no statistically significant differences between the activity estimates obtained using the disclosed method and that the activity estimates obtained using a true attenuation map. Additionally, the disclosed method significantly outperformed the Chang's AC method.

Attenuation compensation (AC) is a pre-requisite for reliable quantification and beneficial for visual interpretation tasks in SPECT imaging. Typical AC methods require the presence of an attenuation map, which is obtained using a transmission scan, such as the CT scan. This has several disadvantages such as increased radiation dose, higher costs, and possible misalignment between SPECT and CT scans. Also, often a CT scan may be unavailable. In this context, the scattered photons in SPECT potentially contain information to estimate the attenuation distribution. To exploit this observation, we developed a simple physics and learning-based method that uses the SPECT emission data in the photopeak and scatter windows to perform transmission-less AC in SPECT. In this example, the method was developed in the context of quantitative 2-D dopamine-transporter (DaT)-scan SPECT imaging.

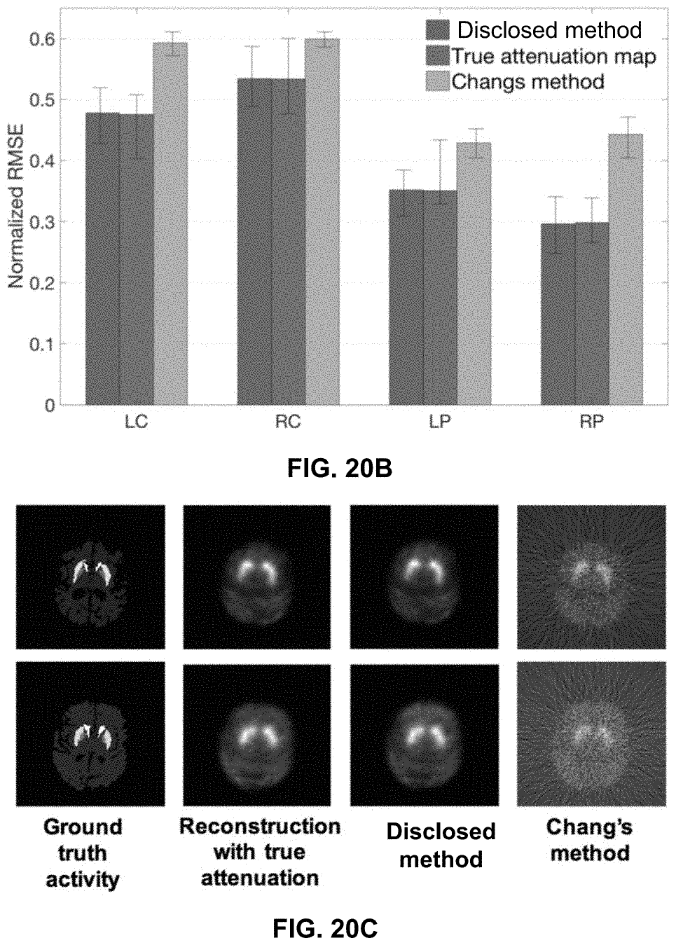

The disclosed method uses data acquired in the scatter window to reconstruct an initial estimate of the attenuation map using a physics-based approach. An auto-encoder is then trained to segment this initial estimate into soft tissue and bone regions. Pre-defined attenuation coefficients are assigned to these regions, yielding the reconstructed attenuation map. This is used to reconstruct the activity distribution using an ordered subsets expectation maximization (OSEM)-based reconstruction approach (FIG. 20A).

The results of these experiments showed no statistically significant differences between the activity estimates obtained using the disclosed method and that with true attenuation map (FIG. 20B). Additionally, the disclosed method significantly outperformed the Chang's AC method. Visually, the images generated using disclosed approach looked similar to those with the true attenuation map and less noisy than those reconstructed with the Chang's AC method (FIG. 20C).

### Example 4: List-Mode OSEM-Based Attenuation and Scatter Compensation Method for SPECT

The following example describes a reconstruction method that uses the entire SPECT emission data, i.e. data in both the photopeak and scatter windows, acquired in list-mode format and including the energy attribute of the detected photon, to perform ASC. The method was implemented using GPU-based hardware and incorporating an ordered subsets expectation maximization (OSEM) algorithm.

Single-photon emission computed tomography (SPECT) has an important role in the diagnosis and therapy of several diseases such as Parkinson's disease, coronary artery dis-ease, and many cancers. A major image-degrading process in SPECT is the scatter and resultant attenuation of photons as they traverse through the tissue before they reach the detector. Reliable attenuation and scatter compensation (ASC) is a pre-requisite for quantification tasks, such as quantifying biomarkers from SPECT images or performing SPECT-based dosimetry. Also, ASC has been observed to be beneficial for visual interpretation tasks. Thus, there is an important need for reliable ASC methods.

Existing ASC methods make use of attenuation maps available from a transmission scan, typically a CT scan. However, typically existing methods do not use the precise value of the energy attribute of the detected photon, as is available when the data are stored in list-mode (LM) format. Using this precise value provides an avenue to improve the ASC. This was a major motivation for ASC approaches based on extensive spectral analysis and modeling. Prior work in PET imaging has shown that using LM data and incorporating energy information led to improved ASC. SPECT LM emission data containing the energy attribute have been observed to contain information to jointly estimate the activity and attenuation distributions. Several studies have also shown that LM data can yield improved reconstruction and quantification compared to binned data in SPECT imaging. These investigations motivate the use of SPECT LM data containing the energy attribute for ASC.

Existing SPECT reconstruction methods also typically use only the photo-peak (PP) window data for estimating the activity. In this context, recent studies have shown that addition of data from the scatter window can provide more information to estimate the activity uptake compared to using data from only the PP window. Using data from PP and scatter windows for activity estimation also increases the effective sensitivity of SPECT systems that are otherwise typically low (<100 counts/million emitted counts). Without being limited to any particular theory, it is thought that processing the entire SPECT data from PP and scatter windows in LM format containing the energy attribute can provide improved quantification compared to using binned data or using only PP window data.

We developed a method to perform ASC using the SPECT emission data acquired in LM format and containing the energy attribute. The method makes use of a previously-published LM reconstruction approach for PET imaging that extends upon previous theory for jointly estimating the activity and attenuation distribution from SPECT LM data. We developed this theory specifically for estimating the activity distribution with known attenuation and derived a maximum-likelihood expectation-maximization (MLEM) algorithm for this task. An ordered-subsets version of this algorithm was then developed and implemented on GPU-based hardware for computational efficiency. The method was objectively evaluated using simulation studies in the context of a quantitative 2D dopamine transporter (DaT)-scan SPECT study.

### Methods

### Theory

Consider a preset-time scintillation-detector-based SPECT system imaging an activity distribution denoted by the vector f. The system acquires and stores data in LM format over a fixed acquisition time, T. Let J denote the number of detected events. Note that the disclosed technique is also applicable to a preset-count system.

Denote attributes collected for the j^{th} LM event by the attribute vector Âⱼ. This vector contains attributes such as the position of interaction with the scintillator, energy deposited in the scintillator, time of interaction, and the angular orientation of the detector that interacted with the photon. Denote the full LM dataset as a set of attribute vector [symbol] ={Âⱼ, j=1, 2, . . . J}. Since the detected LM events are independent, the LM data likelihood is given by

$$pr ( \hat{𝒜} , J \vert f ) = Pr ( J \vert f ) {{\prod }^{J}}_{j = 1} pr ( {\hat{A}}_{j} \vert f ) \qquad Eqn . ( 37 )$$

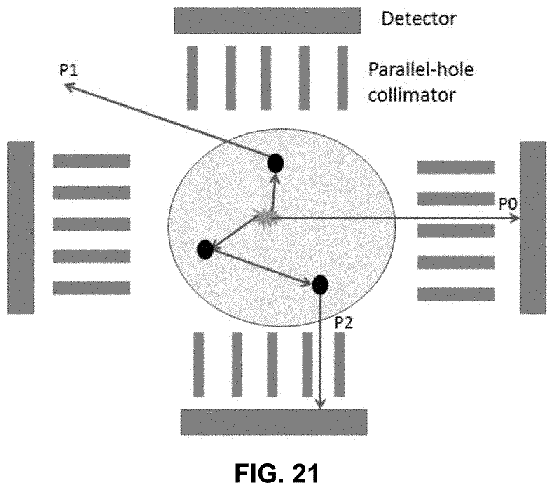

Our approach to developing the reconstruction technique is to estimate f that maximizes the probability of [symbol] . While obtaining the expression for Pr(J|f) is easy since J is Poisson distributed, deriving an expression for pr(Âⱼ|f) is complicated. To address this issue, we use the fact that each detected photon traverses through a specific discrete sub-unit of space after being emitted. We refer to this sub-unit as a path. For example, in FIG. 21, P1, P2, and P3 denote 3 such paths. Note that we do not know in advance of the path that a photon takes. To address this issue while deriving our reconstruction method, we define a latent variable zⱼ [symbol] as follows:

$${z}_{j , ℙ} = \{ \begin{matrix}1 if event j took the path ℙ \\ 0 otherwise .\end{matrix}$$

Defining this latent variable enables developing an expectation-maximization (EM) technique to perform the reconstruction.

Further, it enables deriving the expression for pr(Âⱼ| [symbol] ). More specifically, we can expand the term in Eqn. (37) in terms of a mixture model as:

$$pr ( {\hat{A}}_{j} \vert f ) = {\sum }_{ℙ} pr ( {\hat{A}}_{j} \vert ℙ ) Pr ( ℙ \vert f ) \qquad Eqn . ( 38 )$$

The number of detected events J is Poisson distributed with mean βT where β is the mean rate of detected photons. Using this fact and starting from Eqn. (37), we can write the log-likelihood of the acquired LM data, denoted by [symbol] (f| [symbol] , J̅) as

$$ℒ ( f \vert \hat{𝒜} , J ) = {\sum }_{j = 1}^{J} log {\sum }_{ℙ} pr ( {\hat{A}}_{j} \vert ℙ ) Pr ( ℙ \vert f ) + J log ( β T ) - β T - J \text{?} \\ \text{?} \text{indicates text missing or illegible when filed} \qquad Eqn . ( 39 )$$

The term Pr( [symbol] |f) represents the radiation that is transmitted through the path [symbol] . The expression for this term is given by:

$$Pr ( ℙ \vert f ) = \frac{f ( ℙ ) {s}_{eff} ( ℙ )}{{\sum }_{{ℙ}^{′}} f ( {ℙ}^{′} ) {s}_{eff} ( {ℙ}^{′} )} \qquad Eqn . ( 40 )$$

where denotes f( [symbol] ) the activity of the starting voxel of the path, [symbol] .

The term s_{eff}( [symbol] ) is independent of object activity and models the sensitivity of the path to the detector surface. This term models the attenuation and scatter of photons in the tissue as well as the transmission of photons through the collimator. Using these expressions, we can derive the log-likelihood of the observed LM data and the latent variables, denoted by [symbol] _{C}, as

$${ℒ}_{C} ( f \vert {\{ {\hat{A}}_{j} , {z}_{j} \}}_{j = 1 , 2 … J ,} J ) = {\sum }_{j = 1}^{J} [ {\sum }_{ℙ} {z}_{j , ℙ} \{ log pr ( {\hat{A}}_{j} \vert ℙ ) + log f ( ℙ ) + log {s}_{eff} ( 1 - T \text{?} f ( ℙ ) {s}_{eff} ( ℙ ) . \\ \text{?} \text{indicates text missing or illegible when filed} \qquad Eqn . ( 41 )$$

Having defined the log-likelihood, we develop the LM MLEM (LM-MLEM) technique. In the expectation (E) step of iteration (t+1), we take the expectation of Eqn. (41) conditioned on observed data using the previous estimate of object activity distribution, f^{(t)}. The result is equivalent to replacing [symbol] in Eqn. (40) with its expected value conditioned on the observed data. In the maximization (M) step, we maximize this conditional expectation of the log likelihood. This yields the following iterative update equation:

$${f}_{q}^{( t + 1 )} = \frac{{\sum }_{j = 1}^{J} {\sum }_{{ℙ}_{q}} {\hat{z}}_{j , {ℙ}_{q}}^{( t + 1 )}}{T {\sum }_{{ℙ}_{q}} {s}_{eff} ( {ℙ}_{q} )} \qquad Eqn . ( 42 )$$

where f_{q} and [symbol] _{q} denote the activity in the q^{th} voxel and the paths that originate from the q^{th} voxel, respectively.

### Ordered-Subset LM MLEM (OS-LM-MLEM)

The method as disclosed above is computationally expensive. To reduce the compute time, we developed an ordered-subsets (OS) version of the disclosed technique and implemented this version on parallelized computing hardware.

Without being limited to any particular theory, the OS technique is widely used to achieve faster convergence of EM-based reconstruction methods. To develop an OS version of the developed LM-MLEM technique disclosed above, we divided the LM data into subsets based on the detector angle of each LM event in a manner similar to conventional OSEM-based algorithms. Within each sub-iteration, an estimate of the activity uptake is reconstructed using all the events in a subset. Denote the subset by the index i. Also, let Sᵢ^{p} and Sᵢ^{j} denote the set of paths that reach the detector and the set of events that are detected at angles that are elements of the subset i, respectively. Then, starting from Eqn. (42), the iterative update corresponding to the i^{th} subset and (t+1)^{th} iteration is derived to be:

$${f}_{q}^{( t + 1 , i )} = \frac{{{\sum }_{j = 1}^{J}}_{j ∈ {S}_{i}^{j}} {\sum }_{{ℙ}_{q} ∈ {S}_{i}^{p}} {\hat{z}}_{j , {ℙ}_{q}}^{( t + 1 , i )}}{T {\sum }_{{ℙ}_{q} ∈ {S}_{i}^{p}} {s}_{eff} ( {ℙ}_{q} )} ; i = 1 , 2 … , {N}_{s} , t = 0 , 1 , … , {N}_{g} - 1 , \qquad Eqn . ( 43 )$$

where N_{g} denotes the number of global iterations and Nₛ is the number of sub-iterations or equivalently the number of subsets.

z̅_{j,P}_{_{q}}^{(t+1,i)} in Eqn. (43) can be computed as follows:

$$\text{?} = \frac{pr ( {\hat{A}}_{j} \vert {ℙ}_{q} ) Pr ( {ℙ}_{q} \vert {f}^{( n )} )}{{\sum }_{{ℙ}^{′}} Pr ( {\hat{A}}_{j} \vert {ℙ}^{′} ) pr ( {ℙ}^{′} \vert {f}^{( n )}} \\ \text{?} \text{indicates text missing or illegible when filed} \qquad Eqn . ( 44 )$$

where η is a 2-D vector denoting the iteration state given by

$$η = \{ \begin{matrix}( t , {N}_{s} ) , if i = 0. \\ ( t + 1 , i - 1 ) , otherwise\end{matrix} \qquad Eqn . ( 45 )$$

At any iteration, the number of calculations scales linearly as the number of non-zero voxels, N_{nz}. The reconstruction time was reduced by minimizing N_{nz} in the initial estimate of the activity map. This was done by generating an initial crude estimate of phantom boundary using OSEM reconstruction from a binned sinogram. Further, the computational complexity increases exponentially as the order of scatter. To reduce the number of paths, we considered only up to first-order scatter events and all scatter was assumed to occur in-plane.

To further increase the computational speed, the method was parallelized and implemented on NVIDIA GPU hardware. In each iteration of the OSEM, the evaluation of the denominator of Eqn. (44) and the numerator of Eqn. (43) for a fixed voxel q were performed in parallel using a reduction algorithm.

### Objective Evaluation of Method

The disclosed method was evaluated in the context of a quantitative 2-D DaT-scan SPECT study, where the task was to estimate the mean activity uptake in the caudate and putamen regions of the reconstructed SPECT image. There is much interest in exploring whether uptakes in these regions can help with improved diagnosis of Parkinson's disease. A 2D clinical SPECT scanner with a geometry similar to the Optima 640 parallel-hole collimator and imaging uptake of Ioflupane (I-131) tracer within the brain was simulated. Patient anatomy and physiology were modeled using the Zubal digital brain phantom. The LM data acquisition was modeled using a Monte Carlo-based code, where for each photon, we collected the position of interaction, energy of the photon, and the angular orientation of the detector. To simulate a low-dose setting, we collected around one-third of the number of photons typically acquired clinically.

The disclosed OS-LM-MLEM reconstruction method was used to estimate the activity map. The experiments were repeated for multiple noise realizations. The normalized root-mean-square error (RMSE) of the estimated activity uptakes were computed.

We evaluated the effect of including data from the scatter window on estimating the activity map. For this purpose, we considered three configurations, namely, using data only from the PP window, using data only from photons with energies higher than 120 keV, and using data from the entire energy spectrum.

We also evaluated the effect of binning the energy and position attributes of the LM data on quantification performance. The position attribute was binned into 64 bins and the energy attribute into 2 and 3 bins in different experiments.

### Results

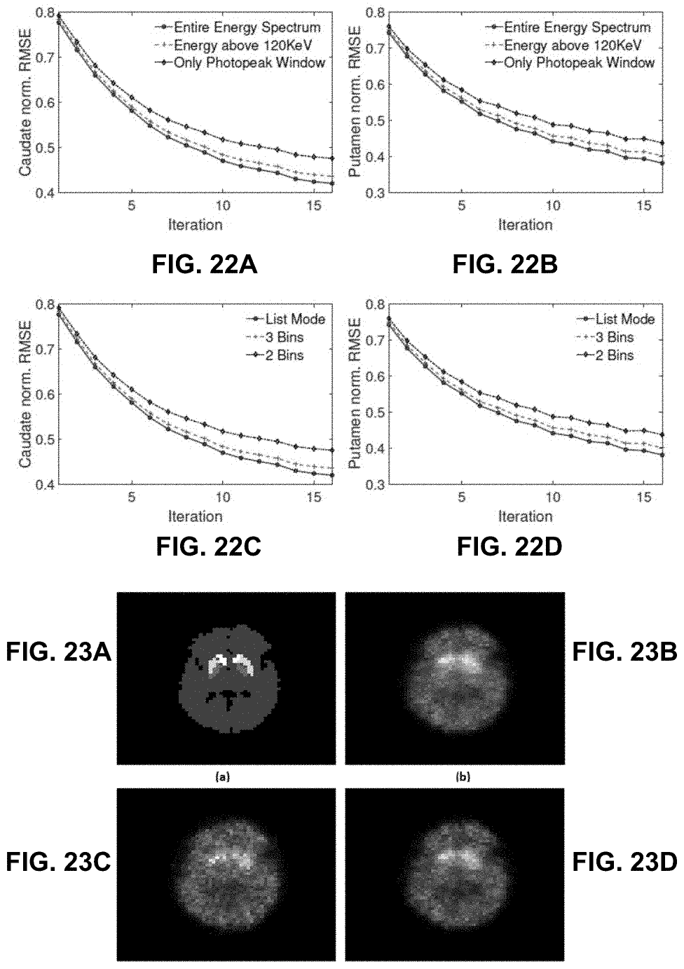

In FIGS. 22A, 22B, 22C, and 22D, the normalized RMSE plots for the activity uptake in the caudate and putamen regions are shown as a function of iteration for the different configurations. FIGS. 22A and 22B show that as we increased the range of energies considered, the RMSE reduced. For example, including data from the entire energy spectrum resulted in approximately 5% decrease in the RMSE for activity uptake in both caudate and putamen compared to using only data from PP window. FIGS. 22C and 22D show that using LM data yielded lower RMSE for both caudate and putamen regions. For example, when using LM data, the RMSE of activity uptake decreased by 6% in caudate and 5% in putamen compared to using two energy bins. Representative reconstructed images with different configurations are shown in FIGS. 23B, 23C, and 23D along with a map of true activity (FIG. 23A). Visually, the results with LM data and using the entire energy spectrum (FIG. 23B) appear to have improved quality. Overall, these results demonstrate that data processed in LM format and encompassing all the emission and scattered photons yielded superior performance.

A pure LM-MLEM based technique was also developed and compared to the OS-LM-MLEM technique (data not shown). We found that the reconstruction results were similar and as expected, the OSEM technique with four subsets yielded a nearly four-fold acceleration in computational speed.

### Conclusions

We disclosed an OS-LM-MLEM-based reconstruction method that uses both the scattered and photo-peak data acquired in LM format to perform ASC in SPECT. Results from realistic simulation studies conducted in the context of measuring regional activity uptakes in a 2-D DaT-scan SPECT study demonstrated that inclusion of data in the scatter window yielded improved quantification compared to using only PP data. Further, processing data in LM format yielded improved quantification compared to binning the energy and position attributes. In various aspects, the disclosed method may be modified to provide for 3D imaging. In various other aspects, the disclosed method may be modified for use with myocardial perfusion SPECT as well as other clinical SPECT imaging applications.

### Example 5: Quantitative Brain SPECT Imaging to Measure Severity of Parkinson's Disease

Quantification errors in SPECT arise due to the image-degrading processes such as low resolution, scatter and resultant attenuation, and noise. Thus, there is an important need to develop methods that can compensate for these sources of errors. This becomes even more vital for small regions such as globus palidus (GP) and substantia nigra (SN) in the brain, which are located deeper in the brain and small, and thus more significantly affected by these artifacts. This has been a major reason for why these structures have not been previously analyzed from brain SPECT images. These key challenges in SPECT quantitation will be addressed using the methods disclosed above that address these artifacts to provide for quantitative brain SPECT.

Reliable quantitation from SPECT projection data requires attenuation and scatter compensation (ASC). The significance of reliable ASC is evident from a previously-published study conducted in 10 patients with PD that observed when images were reconstructed with the typically used Chang's attenuation protocol, there was almost no correlation between the specific binding potential (SBP) and the UPDRS scores (ρ=−0.18). However, when the data were reconstructed with an ASC technique, strong correlation was observed between measured SBP and UPDRS scores (ρ=−0.70, p-value <0.05). However, reliable ASC requires an attenuation map, which is typically obtained from a CT scan. Often, such a CT scan is unavailable. Around 80% of SPECT scanners are single modality and not SPECT/CT. Even when SPECT/CT scanners are available, important concerns related to radiation dose, increased costs, and misalignment between the SPECT and CT scans remain. The ASC method described above in Example 3 overcomes these challenges.

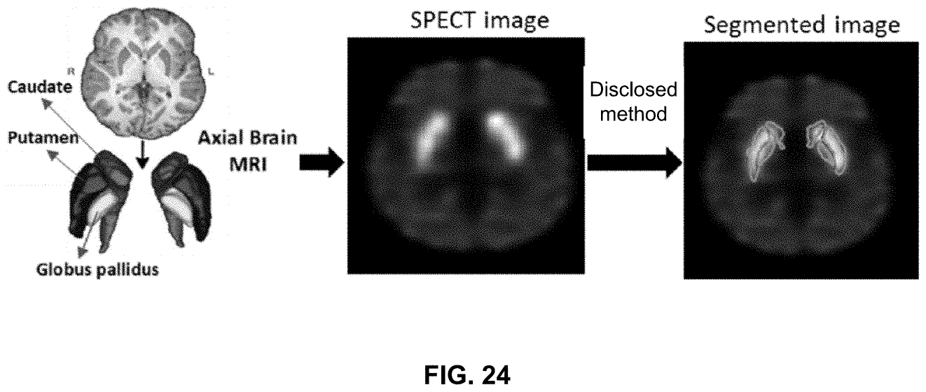

To measure regional DaT uptakes from GP, caudate and putamen, we need to define the boundaries of these structures on SPECT images. These boundaries also assist in compensating for partial volume effects (PVEs) while performing quantitation. Segmentation using existing methods is challenging due to the low resolution of SPECT images and the small size of these regions. FIG. 24 shows how the caudate, putamen, and GP appear as just one region in the SPECT image. Typically, existing segmentation methods and even trained human readers segment these three structures as just one or two regions. These manual and semi-automated segmentations also suffer from significant inter and intra-reader variability, reducing the clinical value of the regional uptake metric. The segmentation methods described in Examples 1, 2, and 4 can delineate these regions accurately and are fully automated, yield reliable DaT uptake values, and provides for quantitative brain SPECT imaging.

Using the segmentation methods described above to obtain reliable DaT uptake values from brain regions previously unable to be segmented will be used to reliably measure the underlying pathophysiology of Parkinson's disease.

The above non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

The patent or application file contains at least one drawing executed in color. Copies of this patent or patent application publication with color drawing(s) will be provided by the Office upon request and payment of the necessary fee.

What is claimed is:

1. A computer-implemented method for segmenting a nuclear medicine image, the method comprising transforming, using a computing device, a nuclear medicine image dataset into a segmented nuclear medicine image dataset using a deep learning network, wherein the segmented nuclear medicine image dataset comprises a plurality of voxels, each voxel associated with at least one voxel volume fraction, each voxel volume fraction indicative of a fraction classified as one tissue type within each voxel.

2. The method of claim 1, wherein the deep learning network is configured to minimize a binary cross-entropy (BCE) of a Bayesian cost function to estimate the posterior mean of the one tissue type within each voxel.

3. The method of claim 2, wherein the deep learning network comprises an autoencoder-decoder architecture.

4. The method of claim 3, further comprising training, using the computing device, the deep learning network using a training dataset comprising a plurality of segmented MRI images and a corresponding plurality of segmented nuclear medicine images.

5. The method of claim 4, wherein the nuclear medicine image is selected from the group consisting of a PET image and a SPECT image.

6. A computer-implemented method for performing transmission-less attenuation and scatter compensation (ASC) on a nuclear medicine image, the method comprising:
a. reconstructing, using a computing device, a scatter window dataset corresponding to the nuclear medicine image to obtain a preliminary attenuation map;
b. transforming, using the computing device, the preliminary attenuation map to a final estimated attenuation map by segmenting the preliminary attenuation map using a deep learning network; and
c. reconstructing, using the computing device, the nuclear medicine image based on a photopeak window associated with the nuclear medicine image in combination with the final estimated attenuation map.

7. The method of claim 6, wherein the deep learning network is configured to minimize a binary cross-entropy (BCE) of a Bayesian cost function to estimate the posterior mean of the one tissue type within each voxel of the preliminary attenuation map.

8. The method of claim 7, wherein the deep learning network comprises an autoencoder-decoder architecture.

9. The method of claim 8, further comprising training, using the computing device, the deep learning network using a training dataset comprising a plurality of segmented MRI images and a corresponding plurality of segmented nuclear medicine images.

10. The method of claim 9, wherein the nuclear medicine image is selected from the group consisting of a PET image and a SPECT image.
